(19) United States
(12) Patent Application Publication
(10) Pub. No.: US 2024/0245728 A1
(43) Pub. Date: Jul. 25, 2024

(54) COMPOSITIONS AND METHODS FOR SPATIAL AND TEMPORAL CONTROL OF NEUROTROPHIC GROWTH FACTOR EXPRESSION

(71) Applicants: THE TRUSTEES OF THE UNIVERSITY OF PENNSYLVANIA, Philadelphia, PA (US); The United States Government as represented by the Department of Veterans Affairs, Washington, DC (US)

(72) Inventors: Daniel Kacy Cullen, Media, PA (US); Hannah Hoeun Lee, Bala Cynwyd, PA (US); Robert Shultz, Philadelphia, PA (US); Sam Lien, Cooper City, FL (US)

(21) Appl. No.: 18/417,617

(22) Filed: Jan. 19, 2024

Related U.S. Application Data
(60) Provisional application No. 63/440,079, filed on Jan. 19, 2023.

(51) Int. Cl. A61K 35/30; A61P 25/02; C07K 14/475; C12N 15/86

(52) U.S. Cl. CPC A61K 35/30; A61P 25/02; C07K 14/4756; C12N 15/86; C12N 2750/14143

(57) ABSTRACT

Provided herein are living scaffolds for peripheral nerve regeneration, the living scaffolds including tissue engineered nerve grafts (TENGs) programmed to express neurogenic growth factors with temporal and spatial control. Further provided herein are compositions and methods for facilitating nerve regeneration, such as compositions and methods comprising TENGs modified to express a neurotrophic growth factor.

### CROSS REFERENCE TO RELATED APPLICATION

This application claims priority under 35 U.S.C. 119(c) to U.S. Provisional Patent Application No. 63/440,079, filed Jan. 19, 2023, which is hereby incorporated herein by reference in its entirety.

### REFERENCE TO SEQUENCE LISTING

This disclosure contains one or more sequences in a computer readable format in an accompanying file titled “046483-7418US1_Sequence_Listing.xml”, which is 29.8 KB in size and was created on Jan. 19, 2024, the contents of which are incorporated herein by reference in their entireties.

### BACKGROUND OF THE INVENTION

Peripheral nerve injuries (PNIs) are common, with approximately 50,000-100,000 patients annually undergoing surgical procedures to address such injuries in the United States and Europe. Despite the advances in microsurgical techniques and nerve repair methods, the rehabilitation course is challenging for patients, and full functional recovery is rarely achieved. In a meta-analysis by Ruijs et al, only 42.6% of patients achieved satisfactory sensory outcome, and 51.6% achieved satisfactory motor outcome. See Ruijs et al., Plast Reconstr Surg. 2005; 116:484-494. Long segment nerve loss, which does not allow for tension-free primary repair, requires grafting procedures, with autograft being the gold standard for a defect greater than 3-cm. However, autografts are associated with donor site morbidity, increased operative time, and most importantly, limited availability. Alternative scaffold options include hollow nerve guidance tubes (NGTs) for shorter gaps and acellular nerve allografts (ANAs) from a cadaver for longer gaps. However, these options remain inferior to autografts in long nerve gap models.

Critically, full recovery is considered impossible for major PNI—those injuries in which a large segment of a nerve is lost (i.e., ≥5-cm) or more proximal injuries (e.g., brachial plexus) that require long distances for axonal regeneration to distal targets (e.g., hand). Hence, patients who experience major PNI face the high probability of a residual functional deficit, even following state-of-the-art surgical reconstruction. One of the major barriers to a good functional outcome after a peripheral nerve repair using conduits or ANAs is insufficient biologically active guidance cues along the length of the scaffold. Axons from the proximal nerve stump have to travel down and reconnect, not only to the distal end, but also through the entire distal nerve segment to end targets (e.g., muscle), in an organized, timely manner, to prevent irreversible end target muscle atrophy. However, current repair strategies fail to overcome the prolonged periods of target muscle denervation, due to the long regenerative distances and slow axonal regeneration rate (˜1-mm/day). Hence, repair and regeneration following PNI is a race against time.

Accordingly, there is a need in the art for articles and methods that guide the nerve ends to regenerate in an accelerated manner to achieve a good functional recovery. The present invention addresses this need.

### BRIEF SUMMARY

In one aspect, the present disclosure generally relates to a composition comprising tissue engineered nerve grafts (TENGs) modified to controllably express a neurotrophic growth factor. In some aspects, the TENGs are modified to controllably express the neurotrophic growth factor under the control of an inducible promoter. In some aspects, the inducible promoter is an inducible tetracycline response element (TRE) promoter. In some aspects, the TENGs are modified to controllably express the neurotrophic growth factor at a first end of the TENG. In some aspects, the TENGs are modified to controllably express the neurotrophic growth factor at a second end of the TENG. In some aspects, the TENGs are modified to controllably express two different neurotrophic growth factors. In some aspects, the TENGs are modified to controllably express a neurotrophic growth factor at a first end of the TENG, and to controllably express a neurotrophic growth factor at a second end of the TENG. In some aspects, the neurotrophic growth factor at the first end of the TENG and the neurotrophic growth factor at the second end of the TENG are different neurotrophic growth factors. In some aspects, the TENGs comprise an expression vector to controllably express the neurotrophic growth factor. In some aspects, the expression vector is delivered by an adeno-associated viral (AAV) vector. In some aspects, the AAV vector is an AAV2 vector. In some aspects, the TENGs comprise a nucleic acid comprising the sequence of SEQ ID NO: 3 or SEQ ID NO: 4. In some aspects, the neurotrophic growth factor is selected from the group consisting of brain-derived neurotrophic factor (BDNF), nerve growth factor (NGF), and glial cell line-derived neurotrophic factor (GDNF). In some aspects, the neurotrophic growth factor is GDNF. In some aspects, the TENGs are stretch-grown TENGs.

Furthermore, in one aspect, the present disclosure generally relates to a method of treating a nerve injury in a subject, wherein the method comprises contacting a site of nerve injury with a stretch-grown tissue engineered nerve graft (TENG) modified to controllably express a neurotrophic growth factor. In some aspects, the subject is a human. In some aspects, the nerve injury comprises an injury to a peripheral nerve of the subject. In some aspects, the nerve injury comprises the loss of a segment of nerve. In some aspects, the nerve injury comprises a nerve lesion of from about 1 cm to about 5 cm in length. In some aspects, the TENGs are modified to controllably express the neurotrophic growth factor under the control of an inducible promoter. In some aspects, the TENGs are modified to controllably express the neurotrophic growth factor at a distal position of the nerve injury. In some aspects, the TENGs are modified to controllably express the neurotrophic growth factor at a proximal position of a nerve injury. In some aspects, the TENGs are modified to controllably express two different neurotrophic growth factors. In some aspects, the TENGs are modified to controllably express a neurotrophic growth factor at a distal position of a nerve injury, and to controllably express a neurotrophic growth factor at a proximal position of a nerve injury. In some aspects, the neurotrophic growth factor at the distal position of the nerve injury and the neurotrophic growth factor at the proximal position of the nerve injury are different neurotrophic growth factors. In some aspects, the TENGs comprise an expression vector to controllably express the neurotrophic growth factor. In some aspects, the expression vector is delivered by an adeno-associated viral (AAV) vector. In some aspects, the neurotrophic growth factor is selected from the group consisting of brain-derived neurotrophic factor (BDNF), nerve growth factor (NGF), and glial cell line-derived neurotrophic factor (GDNF). In some aspects, the neurotrophic growth factor is GDNF.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 presents a schematic representation of exemplary factors that prevent successful peripheral nerve repair.

FIG. 2 presents a schematic representation of the generation of tissue engineered nerve grafts (TENGs) via an exemplary stretch-growth process (see, for instance, Smith, D. H., et al., Sci Adv. 2022; 8:eabm3291). (1) dorsal root ganglia (DRG) neurons are cultured on 2 overlapping membranes, (2) which are gradually pulled apart in custom mechanobioreactors to induce stretch-growth of the spanning axons. (3) Stretch growth occurs for days to weeks at 1 to 10 mm/day. Once the desired axon length is reached, the neurons and stretch-grown axons are encapsulated in collagen extracellular matrix (ECM) for stabilization. (4) Encapsulated constructs are “rolled” and (5) inserted into an NGT or nerve wrap to bridge segmental nerve defects.

FIG. 3A presents a schematic representation of an exemplary tissue engineered nerve graft (TENG).

FIG. 3B presents a schematic representation of regeneration of host axons grown along TENG axons.

FIG. 4A-FIG. 4C present images of the differential expression of GFP and mCherry at each end of a 5 cm TENG. FIG. 4A presents an image of the 5 cm long TENG expressing GFP (left end) and mCherry (right end) at each of the respective ends. FIG. 4B presents an image the left side of the TENG transduced with AAV2-GFP. FIG. 4C presents an image of the right side of TENG transduced with AAV2-mCherry.

FIG. 5A-FIG. 5E present images and graphical representation of results related to the differential expression of GFP and GDNF at each end of a 5 cm TENG. FIG. 5A presents a brightfield image of the GFP side of the 5 cm TENG. FIG. 5B presents a brightfield image of the GDNF side of the 5 cm TENG. FIG. 5C presents an image of AAV2-GFP expression on the GFP side of the TENG. FIG. 5D presents an image of AAV2-GDNF expression on the GDNF side of the TENG. FIG. 5E presents a graphical representation of the hGDNF concentrations of the AAV2-GFP group and the AAV2-GDNF group. Further referring to FIG. 5E, n=6/group, and p<0.001.

FIG. 6 presents a graphical representation of doxycycline-based induction of transgene expression for DRG explants transduced with AAV2-TRE-hGDNF. hGDNF concentration is plotted for the no doxycycline and doxycycline groups. n=3/group, and p=0.039.

FIG. 7 presents a schematic representation of the generation of AAV TENGs.

FIG. 8A-FIG. 8B present images and graphical representations of results related to temporal control of AAV2-GDNF TENGs. FIG. 8A presents an image of an AAV-TENG stretched to 1.5 cm in length, with AAV2-CMV-mCherry on the left end, and AAV2-TRE-GDNF on the right end. FIG. 8B presents a graphical representation of the daily hGDNF concentration plotted against the time in days. Further referring to FIG. 8B, n=4/group, and p=0.013.

FIG. 9A-FIG. 9B present images and graphical representations of results related to spatial and temporal control of AAV2-GDNF TENGs. FIG. 9A presents an image of an AAV-TENG stretched to 1.5 cm in length, with AAV2-CMV-mCherry on the left end, and AAV2-TRE-GDNF on the right end. FIG. 9B presents a graphical representation of the hGDNF concentration for AAV2-CMV-mCherry and AAV2-TRE-hGDNF. Further referring to FIG. 9B, n=3/group, and p=0.034.

FIG. 10 provides a schematic representation of an example of an in vivo study strategy using a rat sciatic nerve injury model.

FIG. 11A-FIG. 11B presents graphical representations of results related to muscle response at 12 weeks (FIG. 11A) and 24 weeks (FIG. 11B) post-repair for control TENG and AAV2-GDNF TENG groups.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

That the disclosure may be more readily understood, select terms are defined below.

As used herein, the terms “a”, “an”, or “the” are used to include one or more than one unless the context clearly dictates otherwise. By way of example, “an element” means one element or more than one element. The term “or” is used to refer to a nonexclusive “or” unless otherwise indicated. The statement “at least one of A and B” or “at least one of A or B” has the same meaning as “A, B, or A and B.”

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

The term “therapeutic” as used herein means a treatment and/or prophylaxis. A therapeutic effect is obtained by any degree of suppression, remission, or eradication of a disease state.

To “treat” a disease as the term is used herein, means to reduce the frequency or severity of at least one sign or symptom of a disease or disorder experienced by a subject.

As used herein, to “alleviate” a disease means reducing the severity of one or more symptoms of the disease.

The terms “patient”, “subject”, and “individual” are used interchangeably and are intended to include living organisms that may be subjected to treatment for a given disease, e.g., mammals. A “subject”, “patient”, or “individual”, as used herein, can be a human or non-human mammal. Non-human mammals include, for example, livestock and pets, such as ovine, bovine, porcine, canine, feline, and murine mammals, as well as simian and non-human primate mammals. Preferably, the subject is human.

As used herein, the terms “effective amount” and “therapeutically effective amount” are used interchangeably and refer to the amount required to reduce or improve at least one symptom or change in a clinical marker of a disease relative to an untreated patient. The effective amount of the treatment used for therapeutic treatment of the disease varies depending upon the manner of the specific disorder, condition or disease, extent of the disorder, condition or disease, and administration of the cells, as well as the age, body weight, and general health of the subject. The effective amount is capable of achieving a particular desired biological result and/or provides a therapeutic or prophylactic benefit.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “expression” as used herein is defined as the transcription and/or translation of a particular nucleotide sequence driven by its promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the terms “conservative variation” or “conservative substitution” generally refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, the term “isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

The term “recombinant polypeptide” as used herein is defined as a polypeptide produced by using recombinant DNA methods.

The term “recombinant DNA” as used herein is defined as DNA produced by joining pieces of DNA from different sources.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

By the term “modified” as used herein, is meant a changed state or structure of a molecule or cell of the invention. Molecules may be modified in many ways, including chemically, structurally, and functionally. Cells may be modified through the introduction of nucleic acids.

As used herein, the term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

As used herein, the term “host cell” includes an individual cell or cell culture that can be or has been a recipient of exogenous polynucleotide(s). Host cells include progeny of a single host cell, and the progeny may not necessarily be completely identical (in morphology or in genomic DNA complement) to the original parent cell due to natural, accidental, or deliberate mutation.

As used herein, the term “cylinder” or “cylindrical” includes a surface consisting of each of the straight lines that are parallel to a given straight line and pass through a given curve. In some aspects, cylinders have an annular profile. In some aspects, the cylinder has a cross-section selected from the group consisting of: a square, a rectangle, a triangle, an oval, a polygon, a parallelogram, a rhombus, an annulus, a crescent, a semicircle, an ellipse, a super ellipse, a deltoid, and the like. In some aspects, the cylinder is the starting point of a more complex three-dimensional structure that can include, for example, complex involutions, spirals, branching patterns, multiple tubular conduits, and any number of geometries that can be implemented in computer-aided design, 3-D printing, and/or in directed evolutionary approaches of secretory organisms (e.g., coral), including of various fractal orders.

“Forced aggregation” and “forced cell aggregation” are used interchangeably herein, and refer to a method of forming “aggregates” or “spheres” of neurons by centrifugation in, for instance, inverted pyramidal micro-wells.

“Forced aggregation TENG” and “forced cell aggregation TENG” are likewise used interchangeably to refer to a TENG that is stretch grown from an aggregate or sphere of neurons formed by forced aggregation.

As used herein, “neurological disorder” refers to any disorder of the body's nervous system, which includes the nerves, brain and spinal cord. In certain embodiments, neurological disorders can arise from injury to the nerves, brain, or spinal cord.

As used herein, “neurodegenerative condition” refers to a condition in which there is a loss of structure or function of neurons, including death of neurons. Examples of neurodegenerative conditions include but are not limited to: Alzheimer's disease, Parkinson's disease, Huntington's disease, prion disease, motor neuron diseases, spinocerebellar ataxia, spinal muscular atrophy, amyotrophic lateral sclerosis (ALS), encephalitis, epilepsy, head and brain malformations, and hydrocephalus.

“Tissue engineered axonal tracts” refer to living axonal tracts generated from TENGs, in which the neuronal cell bodies have been severed leaving only axonal tracts. In some aspects, the TENG has been generated from any sub-type of neuron, including but not limited to neurons from the peripheral nervous system (e.g., spinal motor, sensory dorsal root ganglia), central nervous system (e.g., glutamatergic, GABAergic, dopaminergic, serotonergic), and autonomic nervous system (e.g., ganglionic norepinephrinergic, acetycholinergic, or dopaminergic.

As used herein, the term “tissue-engineered nerve graft (TENG)” refers to a transplantable nerve construct or nerve tissue that is generated in culture. “TENG” is used interchangeably herein with the term “stretch-grown TENG.” In some aspects, TENGs are elongated, or stretch-grown, axons and neurons in a three-dimensional matrix. In some aspects, TENGs are elongated neurons, which comprise long integrated axonal tracts spanning two populations of neurons. In some aspects, TENGs are generated via the stretch growth process described in U.S. Pat. No. 6,365,153, which is incorporated herein by reference in its entirety. In some aspects, the elongated neuron is a mechanically elongated neuron or any method that produces elongated tracts of axons. Following mechanical elongation of neurons to produce stretch-grown axons, the cultures can be embedded in a three-dimensional matrix for removal from the culture environment, thus creating “stretch-grown TENGs.” As used herein, TENGs are distinct from “neurons in a three-dimensional matrix,” which are not stretch-grown or elongated. In some aspects, stretch-grown TENGs are transplanted, or connected, to the distal nerve structure following nerve injury and serve to maintain the pro-regenerative capacity of the environment of the denervated distal nerve. In some aspects, neurons in a three-dimensional matrix or in suspension are transplanted to the distal nerve via direct injection into the nerve structure following nerve injury, and serve to maintain the pro-regenerative capacity of the environment of the distal nerve structure. In some aspects, tens of neurons, thousands of neurons, millions of neurons, tens of millions of neurons, or more are injected. In some aspects, multiple direct deliveries and/or injections of neurons are conducted over time. In some aspects, neuron injections are conducted quarterly, bi-annually, annually, or according to various regimens until the nerve injury has been sufficiently treated.

The term “neuron” is used interchangeably herein with the term “neuronal cell.”

As used herein, a “nerve construct” refers to composition comprising at least one neuron.

As used herein, “elongated neuron” is used interchangeably with “stretch-grown neuron” and refers to a neuron that has had the axon increase in length as a result of an ex vivo stretching procedure compared to a comparable neuron that has not been subjected to the ex vivo stretching procedure. In some aspects, an elongated neuron comprises a cell body and at least one elongated axon. In some aspects, the at least one elongated neuron possesses an elongated axon that spans a distance and is connected to another cell, preferably another neuron and more preferably, another elongated neuron. Exemplary stretching procedures are described, for example, in U.S. Pat. No. 6,365,153. In some aspects, the elongated neuron is a mechanically elongated neuron.

As used herein, “biocompatible” refers to a material that is substantially non-toxic to neuronal cell bodies and axons and that is substantially non-toxic to the cells and tissues of a recipient of the composition. A biocompatible material, when introduced into an individual, is not toxic or injurious to that individual, nor does it induce immunological rejection of the material in the mammal.

As used herein, “synapse” refers to a junction between a neuron and another cell, across which chemical communication flows. As used herein, “synapsed” refers to a neuron that has formed one or more synapses with one or more cells, such as another neuron or a muscle cell. As used herein, “synaptically integrate” refers to the formation of at least one synapse between a neuron and at least one other cell. In some aspects, the other cell is a nerve cell, a muscle cell or another neuron target. For instance, two neurons are synaptically integrated if at least one synapse exists between the two cells.

As used herein, a “sheath” refers to a structure intended to support, position and/or hold a nerve in place. In some aspects, a sheath can provide a means of securing the position of a TENG. In some aspects, the sheath is intended to hold a nerve construct in place, for instance, in a nerve lesion site. In some aspects, the sheath is endogenous (e.g., the epineurium or perineurium) or exogenous (e.g., a nerve guidance tube) to the nerve. In some aspects, a sheath at least partially enfolds a nerve or nerve construct. As used herein, “at least partially enfolds” encompasses partial or complete surrounding of part or all of a nerve or nerve construct. It therefore encompasses a completely surrounded nerve or nerve construct. Surrounding a nerve or nerve construct means at least one neuron is surrounded by a sheath. In some aspects, the neuron is all or a part of a stretch-grown nerve construct. In some aspects, the sheath is a synthetic sheath. In some aspects, the synthetic sheath is flexible and can be sutured. Optionally, the sheath is bio-resorbable or biodegradeable.

As used herein, “proximal” refers to a position that is nearer to the spinal cord or brain as compared to another or other positions. As used herein, “distal” refers to a position that is some distance from the primary injury site. The distal position is farther away on the nerve path as compared to the proximal position. The distance between the distal position and the injury site can be any distance including 1 mm or smaller, up to one or more meters in length or more. For example, the distance between the distal position and the injury site can be less than 1 cm, at least about 1 cm, at least about 3 cm, at least about 10 cm, at least about 1 meter, or more. Multiple distal sites are also encompassed by the present disclosure. Thus, in some aspects, stretch-grown TENG or neurons in a three-dimensional matrix or in suspension, or both, can be applied to multiple distal sites in a subject. In some aspects, the proximal or distal position of a nerve injury is referred to as the proximal nerve sheath, stump or structure, or the distal nerve segment, stump or structure, respectively. Thus, in the context of a nerve injury, the terms “distal sheath,” “distal stump,” and “distal structure,” are used interchangeably herein. Similarly, the terms, “proximal sheath,” “proximal stump,” and “proximal structure” are used interchangeably herein.

As used herein, a “neurotrophic factor”, “neurotrophic growth factor”, “neurogenic factor”, and “neurogenic growth factor” are used interchangeably to refer to a biological, recombinant, or synthetic molecule that contributes to the growth and survival of neurons during development, and/or for maintaining adult neurons. Exemplary neurotrophic growth factors include, but are not limited to, neurotrophin (including, for example, neurotrophin 3 (NT-3), neurotrophin 4/5 (NT-4/5), neurotrophin 6 (NT-6), and neurotrophin 7 (NT-7)), ciliary neurotrophic factor (CNTF), brain-derived neurotrophic factor (BDNF), nerve growth factor (NGF), basic fibroblastic growth factor (bFGF), insulin-like growth factor (IGF)-1, glial cell line-derived neurotrophic factor (GDNF), neurturin, artemin, persephin, purpurin, transforming growth factor-beta (TGF-beta), and synthetic neurotrophins, such as pan-neurotrophin-1 (PNT-1).

As used herein, “treating a nerve lesion” is used interchangeably with the term “treating a nerve injury” to refer to repairing the injured nerve region or reducing the frequency and/or the severity of a symptom of the nerve lesion.

As used herein, the terms “nerve injury” and “nerve lesion” are used interchangeably to refer to any damage or disruption of neuronal axons.

As used herein, the term “transplant” refers to making contact between, or connecting, an exogenous cell or tissue and a host cell or tissue. In the context of TENGs, by “transplant” is meant implantation of the TENG at the desired site. In the context of neurons that do not comprise a TENG, by “transplant” is meant administration of neurons to the desired site, for example, by injection. For example, in some instances neurons are injected directly into the distal nerve segment or into one or more conduits/devices that directly interface with the distal sheath. In some aspects, the TENG is transplanted to the site of a nerve injury. In some aspects, the TENG is transplanted to the distal nerve structure. In some aspects, the TENG comprises stretch-grown axons. Methods for transplantation are known to those of skill in the art of cell transplantation. See, for instance, U.S. Pat. No. 6,365,153, incorporated by reference herein in its entirety. Suitable transplant material can be evaluated by using well-known electrophysiological and fluorescence techniques. In some aspects, transplantation of a TENG involves removing a portion of a nerve at the site of transplant and placing the TENG at the site. In some aspects, the TENG comprises a bioresorbable sheath that is sutured to tissue site of transplant in order to secure the position of the TENG. In some aspects, the TENG comprises mechanically elongated neurons from an intended recipient (autologous transplantation), from a genetically identical donor (syngeneic transplantation) or from a non-genetically identical donor (allogeneic or xenogeneic transplantation). In some aspects, transplantation of neurons involves injecting neurons into the distal nerve segment.

As used herein, “in vitro” and “ex vivo” are used interchangeably to refer to conditions outside the body of a living organism. Thus, in vitro culturing and ex vivo culturing both refer to culturing outside the body of a living organism.

As used herein, the term “babysit” or “babysitting” refers to the function of a composition, method, process, or procedure for maintaining the pro-regenerative capacity of the distal nerve segment following a nerve injury. In some aspects, the Schwann cells in the distal nerve segment are maintained in a pro-regenerative phenotype and alignment by compositions or methods that serve to babysit the distal nerve segment. In some aspects, the compositions and methods that serve to babysit the distal nerve segment enhance the survival of Schwann cells. In some aspects, the pro-regenerative capacity of the distal nerve segment is maintained until at least such time as proximal nerve axons reinnervate distal targets.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions and Methods for Spatial and Temporal Control of Neurotrophic Growth Factors for Nerve Regeneration

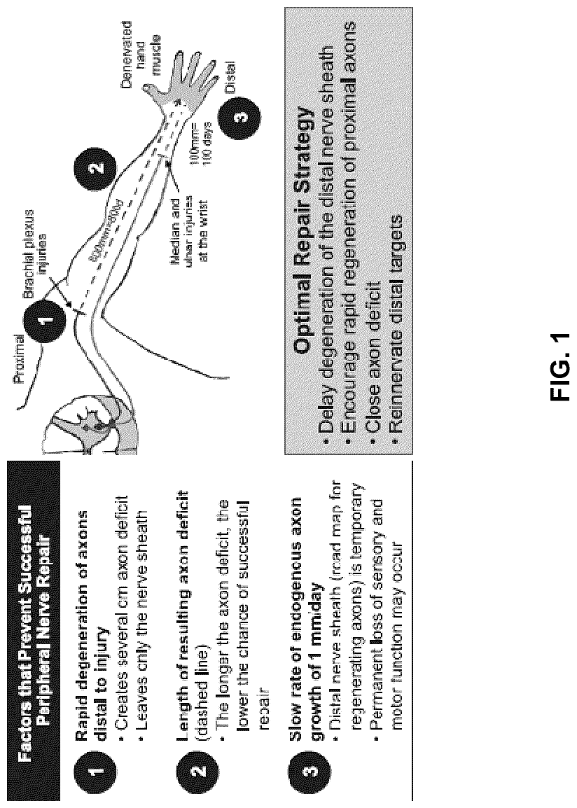

Full recovery is considered nearly impossible for major PNI—those injuries in which a large segment of a nerve is lost (i.e., ≥5-cm), or more proximal injuries (e.g., brachial plexus) that require long distances for axonal regeneration to distal targets (e.g., hand). Hence, patients who experience major PNI face the high probability of a residual functional deficit, even following state-of-the-art surgical reconstruction. One of the major barriers to a good functional outcome after a peripheral nerve repair using conduits or acellular nerve allografts (ANAs) is insufficient biologically active guidance cues along the length of the scaffold. Axons from the proximal nerve stump have to travel down and reconnect, not only to the distal end, but also through the entire distal nerve segment to end targets (e.g., muscle), in an organized, timely manner, to prevent irreversible end target muscle atrophy. However, current repair strategies fail to overcome the prolonged periods of target muscle denervation, due to the long regenerative distances and slow axonal regeneration rate (˜1-mm/day) (FIG. 1). Numerous strategies have been explored to augment expression of these biologically active guidance cues, such as neurogenic growth factors. However, uncontrolled expression of growth factors has been shown to lead to deleterious effect due to axon trapping, known as the “candy store effect”. See, for example, Tannemaat, M. R., Eur J Neurosci. 2008; 28:1467-1479.

Prolonged expression of neurotrophic growth factors is neither necessary nor desirable. For instance, GDNF, while promoting motor neuron survival and axon outgrowth, has been shown to be deleterious when it is expressed in a prolonged and ubiquitous manner. See, for instance, Shakhbazau, A., et al., J Control Release. 2013; 172:841-851; Tannemaat, M. R., Eur J Neurosci. 2008; 28:1467-1479; and Santosa, K. B., et al., Muscle Nerve. 2013; 47:213-223. Such deleterious effects were due in part to aberrant sprouting, axon entrapment, nerve hypertrophy, and weight loss.

As such, the present disclosure generally relates to compositions and methods for facilitating nerve regeneration. In one aspect, the present disclosure relates to a composition comprising tissue engineered nerve grafts (TENGs) modified to controllably express a neurotrophic growth factor. In one aspect, the present disclosure relates to a method of treating a nerve injury in a subject, wherein the method comprises contacting a site of nerve injury with a stretch-grown tissue engineered nerve graft (TENG) modified to controllably express a neurotrophic growth factor.

Using the compositions and methods described herein, a novel living scaffold expressing neurogenic growth factors with temporal and spatial control was developed. In some aspects, the compositions and methods described herein can be used to bridge long segment nerve gaps where autografts have previously been used as autografts have increasingly fallen out of favor due to donor site morbidity, increased operative time, and limited availability.

In some aspects, the compositions and methods described herein can provide different growth factors at the proximal and distal ends of the scaffold for optimal function at each ends. In some aspects, expression of each of the growth factors can be turned off once the nerve regeneration is underway to prevent the candy-store effect of axon trapping. In some aspects, the temporal control comprises use of an inducible element, such as a doxycycline inducible system for expression.

### Compositions

In one aspect, the present disclosure relates to a composition comprising tissue engineered nerve grafts (TENGs) modified to controllably express a neurotrophic growth factor. In some aspects, the composition comprises stretch-grown tissue engineered nerve grafts (TENGs). In some aspects, the composition comprises stretch-grown TENGs expressing a neurogenic growth factor with temporal and spatial control, such as by using adeno-associated virus (AAV) gene transfer technology (AAV-TENGs). In some aspects, the differential spatial distribution of transgene expression for TENGs establishes a gradient of neurotrophic growth factors. In some aspects, external temporal control of the transgene expression is provided through inducible tetracycline response element (TRE) promoter technology and doxycycline, a well-tolerated antibiotic in use clinically. In some aspects, the AAV-TENGs disclosed herein are used to enhance regenerative strategies and/or serve as a platform to investigate the effects of different growth factors in spatially and temporally controlled manner, such as after peripheral nerve injuries (PNIs).

### Neurotrophic Growth Factors and Expression Thereof

In some aspects, the TENGs are modified to controllably express the neurotrophic growth factor at a distal position of a nerve injury. In some aspects, the TENGs are modified to controllably express the neurotrophic growth factor at a first end of the TENG. In some aspects, the nerve injury comprises an injury to a second end of the TENG.

In some aspects, the TENGs are modified to controllably express at least two, e.g., two, different neurotrophic growth factors. In some aspects, the TENGs are modified to controllably express 1, 2, 3, 4, 5, 6, 7, 8, 9, or 10 different neurotrophic factors. In some aspects, the TENGs are modified to controllably express a neurotrophic growth factor at a first end of the TENG, and to controllably express a neurotrophic growth factor at a second end of the TENG. In some aspects, the neurotrophic growth factor at the first end of the TENG and the neurotrophic growth factor at the second end of the TENG are different neurotrophic growth factors. In some aspects, the neurotrophic growth factor is selected from the group consisting of brain-derived neurotrophic factor (BDNF), nerve growth factor (NGF), and glial cell line-derived neurotrophic factor (GDNF). In some aspects, the neurotrophic growth factor is GDNF. In some aspects, the neurotrophic growth factor comprises or is neurotrophin (including, for example, neurotrophin 3 (NT-3), neurotrophin 4/5 (NT-4/5), neurotrophin 6 (NT-6), and neurotrophin 7 (NT-7)), ciliary neurotrophic factor (CNTF), brain-derived neurotrophic factor (BDNF), nerve growth factor (NGF), basic fibroblastic growth factor (bFGF), insulin-like growth factor 1 (IGF-1), glial cell line-derived neurotrophic factor (GDNF), neurturin, artemin, persephin, purpurin, transforming growth factor-beta (TGF-beta), and/or synthetic neurotrophins, such as pan-neurotrophin-1 (PNT-1). In some aspects, the neurotrophic growth factor is selected from the group consisting of brain-derived neurotrophic factor (BDNF), insulin-like growth factor 1 (IGF-1), nerve growth factor (NGF), and glial cell line-derived neurotrophic factor (GDNF).

### Polynucleotides and Vectors

In some aspects, the TENGs comprise an expression vector to controllably express the neurotrophic growth factor. In some aspects, the expression vector is delivered by an adeno-associated viral (AAV) vector. In some aspects, the AAV vector is an AAV2 vector. In some aspects, the TENGs comprise a nucleic acid comprising the sequence of SEQ ID NO: 3 or SEQ ID NO: 4.

In some aspects, the TENGs are modified to controllably express the neurotrophic growth factor under the control of an inducible promoter. In some aspects, the inducible promoter is an inducible tetracycline response element (TRE) promoter. In some aspects, the TENG modified to controllably express a neurotrophic factor comprises any of the control elements described herein, e.g., promoters.

In some aspects, the polynucleotide encoding the neurotrophic growth factor is operably linked to a transcriptional control element, e.g., a promoter, and enhancer, and so forth. Suitable promoter and enhancer elements are known to those of skill in the art. In some aspects, the polynucleotide encoding the neurotrophic growth factor is operably linked to a promoter. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, and so forth, is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), and so forth), tetracycline regulated promoters, (e.g., promoter systems including TetActivators, TetON, TetOFF, and so forth), steroid regulated promoters (e.g., rat glucocorticoid receptor promoter systems, human estrogen receptor promoter systems, retinoid promoter systems, thyroid promoter systems, ecdysone promoter systems, mifepristone promoter systems, and so forth), metal regulated promoters (e.g., metallothionein promoter systems, and so forth), pathogenesis-related regulated promoters (e.g., salicylic acid regulated promoters, ethylene regulated promoters, benzothiadiazole regulated promoters, and so forth), temperature regulated promoters (e.g., heat shock inducible promoters (e.g., HSP-70, HSP-90, soybean heat shock promoter, and so forth), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a tre promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lacl repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters.

Inducible promoters are also contemplated as part of the invention, such as a part of controllable expression of a neurotrophic factor by a modified TENG. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some aspects, the polynucleotide or vector containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, and so forth known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A polynucleotide encoding the neurotrophic growth factor can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLnco, pSV2cat, pOG44, PXRI, pST-Ki, pST-KiT, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some aspects, an expression vector can be used to introduce the neurotrophic growth factor into a cell. Accordingly, in some aspects an expression vector (e.g., a AAV vector, e.g., an AAV2 vector) of the present invention comprises a nucleic acid encoding for the neurotrophic growth factor. In some aspects, the expression vector will comprise additional elements that will aid in the functional expression of the neurotrophic growth factor thereof encoded therein. In some aspects, an expression vector comprising a nucleic acid encoding for the neurotrophic growth factor further comprises a mammalian promoter. In one aspect, the vector further comprises an elongation-factor-1-alpha promoter (EF-1α promoter). Use of an EF-1α promoter may increase the efficiency in expression of downstream transgenes. Physiologic promoters (e.g., an EF-1α promoter) may be less likely to induce integration mediated genotoxicity, and may abrogate the ability of the retroviral vector to transform stem cells. Other physiological promoters suitable for use in a vector are known to those of skill in the art and may be incorporated into a vector of the present invention. In some aspects, the vector further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some aspects, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some aspects a vector for the present invention further comprises a WPRE sequence. Various posttranscriptional regulator elements are known to those of skill in the art and may be incorporated into a vector of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one aspect, a vector of the present invention includes a 3′ U3 deleted LTR. Accordingly, a vector of the present invention may comprise any combination of the elements described herein to enhance the efficiency of functional expression of transgenes.

In some aspects, vectors of the present invention are self-inactivating vectors. As used herein, the term “self-inactivating vector” refers to vectors in which the 3′ LTR enhancer promoter region (U3 region) has been modified (e.g., by deletion or substitution). In some instances, a self-inactivating vector can prevent viral transcription beyond the first round of viral replication. Consequently, a self-inactivating vector is capable in some instances of infecting and then integrating into a host genome (e.g., a mammalian genome) only once, and cannot be passed further. Accordingly, self-inactivating vectors can greatly reduce the risk of creating a replication-competent virus.

In order to assess the expression of the neurotrophic growth factor, the expression vector to be introduced into a cell may also contain either a selectable marker gene or a reporter gene, or both, to facilitate identification and selection of expressing cells from the population of cells sought to be transfected or infected through viral vectors. In some aspects, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### TENGs

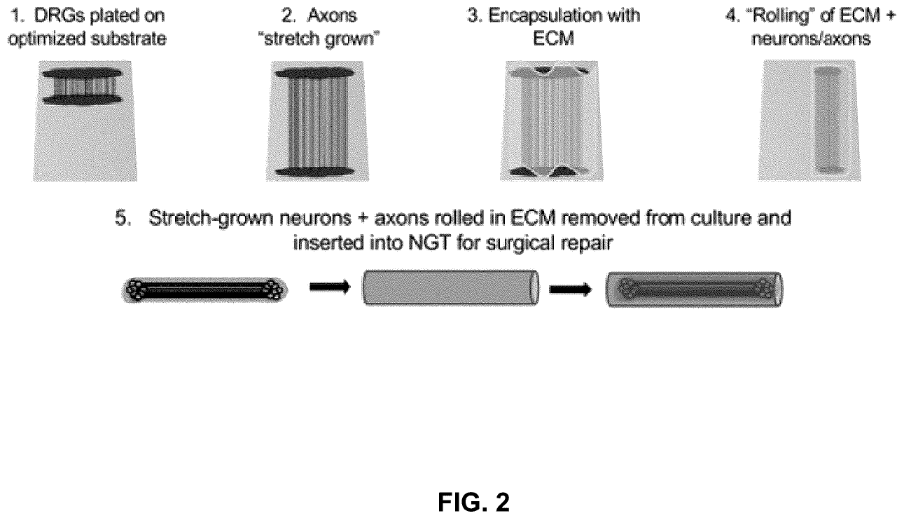

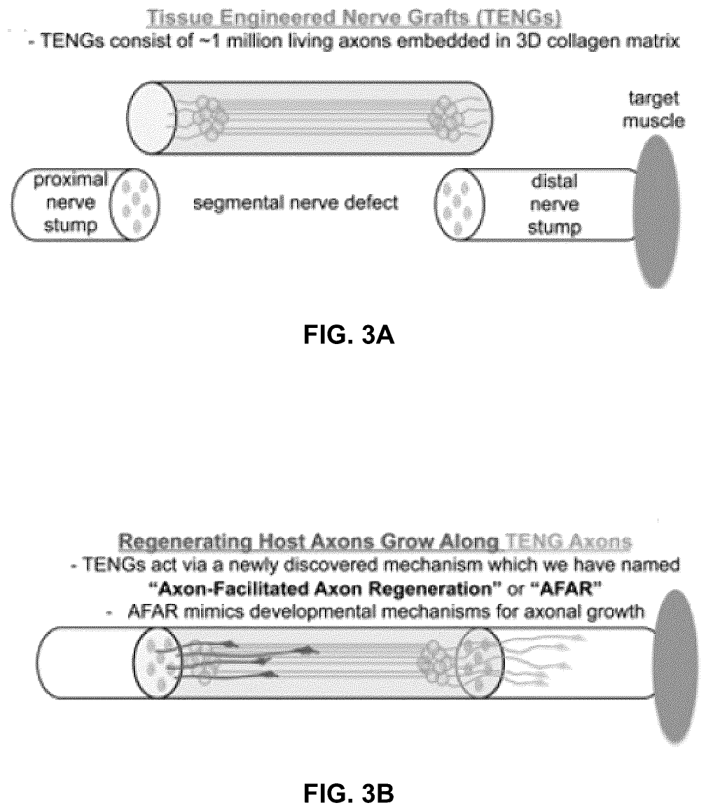

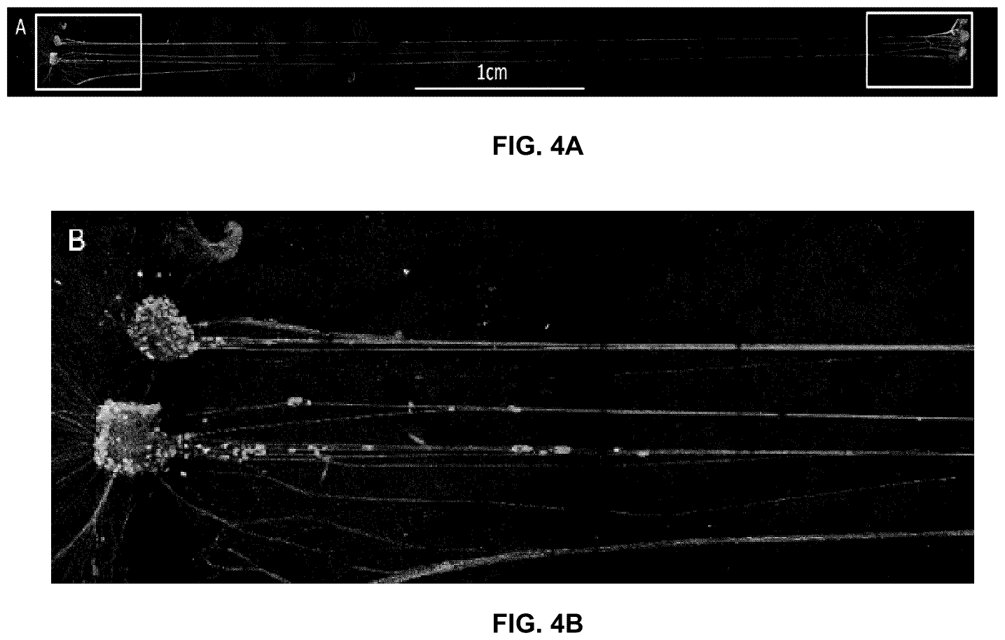

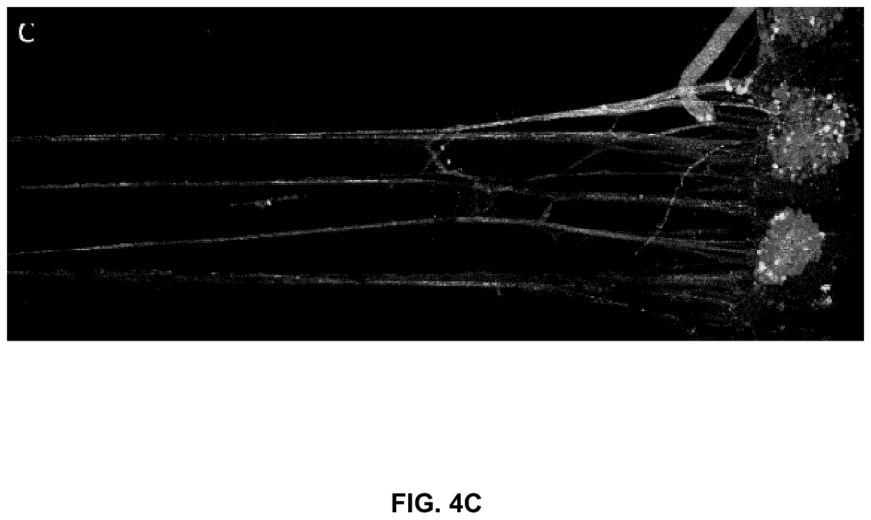

In general, TENGs are living three-dimensional nerve constructs that comprise neurons and longitudinally aligned axonal tracts spanning discrete neuronal populations-thus mimicking aspects of the structure of the lost nerve. The ability to generate TENGs is based upon seminal discoveries regarding axon growth via continuous mechanical tension, also referred to as “stretch growth” (Smith et al., Tissue Eng 7, 131-139 (2001)). Stretch growth is a mechanism that can extend integrated axons (i.e. post-synaptic) at rapid rates using custom mechanobioreactors through controlled separation of two neuron populations. During stretch growth, individual axons gradually coalesce to form large axonal tracts, called fascicles, taking on a highly organized parallel orientation. This process can rapidly generate axon tracts of unprecedented lengths of 5-10 cm in 14-21 days, with no theoretical limit as to the final length (Pfister et al., J Neurosci 24, 7978-7983 (2004); Smith, Prog Neurobiol 89, 231-239 (2009), U.S. Pat. No. 6,365,15) (FIGS. 2A-2D). Three-dimensional TENGS are subsequently created by embedding the living axonal tracts in an extracellular matrix and removing from culture en masse (Pfister et al., J Neurosci Methods 153, 95-103 (2006); Huang et al., Tissue Eng Part A 15, 1677-1685 (2009)). It was demonstrated in rat and pig models of PNI, that allogeneic TENGs possess both bridging and babysitting properties and can overcome inherent limitations of nerve regeneration that cripple current repair options. In some aspects, the TENGs are stretch-grown TENGs. In some aspects, the TENGs comprises TENGs as described in U.S. Pat. No. 10,525,085. In some aspects, the TENGs comprises forced aggregation TENGs as described in U.S. Published App. No. 2020/0230293. In some aspects, the TENGs comprise a first end and a second end. For instance, the left end of the TENG of FIG. 4A could be considered the first end of the TENG, and the right end of the TENG of FIG. 4A could be considered the second end of the TENG, or, alternatively, the right end of the TENG of FIG. 4A could be considered the first end of the TENG, and the left end of the TENG of FIG. 4A could be considered the second end of the TENG.

### Methods

In one aspect, the present disclosure generally relates to a method of treating a nerve injury comprising use of any of the compositions described herein. In one aspect, the present disclosure relates to a method of treating a nerve injury in a subject, wherein the method comprises contacting a site of nerve injury with a stretch-grown tissue engineered nerve graft (TENG) modified to controllably express a neurotrophic growth factor. In some aspects, the subject is a human. In some aspects, the subject is a veterinary animal, such as, but not limited to, non-human primates, horses, cattle, sheep, dogs, cats, pigs, and goats. In some aspects, the subject is a mammal. In some aspects, the nerve injury comprises an injury to a peripheral nerve of the subject. In some aspects, the nerve injury comprises the loss of a segment of nerve. In some aspects, the nerve injury comprises a nerve lesion of from about 1 cm to about 5 cm in length. In some aspects, the nerve injury comprises a nerve lesion of about 0.1, about 0.5, about 1.0, about 1.5, about 2.0, about 2.5, about 3.0, about 3.5, about 4.0, about 4.5, about 5.0, about 6.0, about 7.0, about 8.0, about 9.0, about 10.0, or more centimeters (cm) in length. In some aspects, the nerve injury comprises a nerve lesion of less than 1 cm in length. In some aspects, the nerve injury comprises a nerve lesion of at least about 1 cm in length. In some aspects, the nerve injury comprises a nerve lesion of at least about 3 cm in length. In some aspects, the nerve injury comprises a nerve lesion of at least about 5 cm in length.

In some aspects, the nerve injury is a nerve injury that occurs as a result of trauma, a surgical procedure, the positioning of a patient during surgery, a compression or crush injury, a disease related to a loss of motor or sensory nerve function, a congenital anomaly, an amputation, complete or partial removal of an organ, tumor or tissue, a metabolic/endocrine complication, inflammatory disease, autoimmune disease, vitamin deficiency, infectious disease, toxin, exposure to organic metal or heavy metal, or administration of a medication or drug. In some aspects, the nerve injury is selected from the group consisting of: peripheral nerve injury, brain injury, and spinal cord injury.

In some aspects, the TENGs are modified to controllably express the neurotrophic growth factor under the control of an inducible promoter. In some aspects, the TENGs are modified to controllably express the neurotrophic growth factor at a distal position of the nerve injury. In some aspects, the TENGs are modified to controllably express the neurotrophic growth factor at a proximal position of a nerve injury. In some aspects, the TENGs are modified to controllably express two different neurotrophic growth factors. In some aspects, the TENGs are modified to controllably express a neurotrophic growth factor at a distal position of a nerve injury, and to controllably express a neurotrophic growth factor at a proximal position of a nerve injury. In some aspects, the neurotrophic growth factor at the distal position of the nerve injury and the neurotrophic growth factor at the proximal position of the nerve injury are different neurotrophic growth factors. In some aspects, the TENGs comprise an expression vector to controllably express the neurotrophic growth factor. In some aspects, the expression vector is an adeno-associated viral (AAV) vector. In some aspects, the neurotrophic growth factor is selected from the group consisting of brain-derived neurotrophic factor (BDNF), nerve growth factor (NGF), and glial cell line-derived neurotrophic factor (GDNF). In some aspects, the neurotrophic growth factor is selected from the group consisting of brain-derived neurotrophic factor (BDNF), insulin-like growth factor 1 (IGF-1), nerve growth factor (NGF), and glial cell line-derived neurotrophic factor (GDNF). In some aspects, the neurotrophic growth factor is GDNF. In some aspects, the method promotes axonal regeneration in the treated subject.

In some aspects, the methods provided herein are used in addition to a primary procedure and provide a greater degree of functional recovery following repair of PNI, as compared to the degree of functional recovery that occurs when only the primary procedure is utilized. For example, in some aspects, functional recovery is increased by about 5%, about 10%, about 20%, about 30%, about 40%, about 50%, about 60%, about 70%, about 80%, about 90%, about 100% or more by the use of the methods provided herein. In some aspects, the methods provided herein are used in the absence of a primary procedure and provide a greater degree of functional recovery following repair of PNI.

### Pharmaceutical Compositions

In some aspects, pharmaceutical compositions of the present invention comprise TENGs modified to controllably express a neurotrophic growth factor, such as those described herein, in combination with one or more pharmaceutically or physiologically acceptable carriers, diluents or excipients. In some aspects, such compositions comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some aspects, pharmaceutical compositions of the present invention can be administered in a manner appropriate to the disease to be treated (or prevented). The quantity and frequency of administration will be determined by such factors as the condition of the patient, and the type and severity of the patient's disease, although appropriate dosages may be determined by clinical trials.

In some aspects, the TENGs modified to controllably express a neurotrophic growth factor as described herein to be administered are autologous, allogeneic or xenogeneic with respect to the subject undergoing therapy.

In some aspects, the TENGs modified to controllably express a neurotrophic growth factor as described herein can be administered in dosages and routes and at times to be determined in appropriate pre-clinical and clinical experimentation and trials. In some aspects, compositions are administered multiple times at dosages within these ranges. In some aspects, administration of the cells of the invention is combined with other methods useful to treat the desired disease or condition as determined by those of skill in the art.

In some aspects, the administration of the TENGs modified to controllably express a neurotrophic growth factor as described herein is be carried out in any convenient manner known to those of skill in the art. In some aspects, the TENGs modified to controllably express a neurotrophic growth factor as described herein is administered to a subject by, injection, implantation or transplantation.

The disclosures of each and every patent, patent application, and publication cited herein are hereby incorporated herein by reference in their entirety.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

SEQUENCE TABLE

| SEQ | | |
|---|---|---|
| ID | | |
| NO | DESCRIPTION | SEQUENCE |
| 1 | AAV2-CMV- | CTGCGCGCTCGCTCGCTCACTGAGGCCG |
| | GFP | CCCGGGCAAAGCCCGGGCGTCGGGCGAC |
| | | CTTTGGTCGCCCGGCCTCAGTGAGCGAG |
| | | CGAGCGCGCAGAGAGGGAGTGGCCAACT |
| | | CCATCACTAGGGGTTCCTTCTAGACAAC |
| | | TTTGTATAGAAAAGTTGTAGTTATTAAT |
| | | AGTAATCAATTACGGGGTCATTAGTTCA |
| | | TAGCCCATATATGGAGTTCCGCGTTACA |
| | | TAACTTACGGTAAATGGCCCGCCTGGCT |
| | | GACCGCCCAACGACCCCCGCCCATTGAC |
| | | GTCAATAATGACGTATGTTCCCATAGTA |
| | | ACGCCAATAGGGACTTTCCATTGACGTC |
| | | AATGGGTGGAGTATTTACGGTAAACTGC |
| | | CCACTTGGCAGTACATCAAGTGTATCAT |
| | | ATGCCAAGTACGCCCCCTATTGACGTCA |
| | | ATGACGGTAAATGGCCCGCCTGGCATTA |
| | | TGCCCAGTACATGACCTTATGGGACTTT |
| | | CCTACTTGGCAGTACATCTACGTATTAG |
| | | TCATCGCTATTACCATGGTGATGCGGTT |
| | | TTGGCAGTACATCAATGGGCGTGGATAG |
| | | CGGTTTGACTCACGGGGATTTCCAAGTC |
| | | TCCACCCCATTGACGTCAATGGGAGTTT |
| | | GTTTTGGCACCAAAATCAACGGGACTTT |
| | | CCAAAATGTCGTAACAACTCCGCCCCAT |
| | | TGACGCAAATGGGCGGTAGGCGTGTACG |
| | | GTGGGAGGTCTATATAAGCAGAGCTGGT |
| | | TTAGTGAACCGTCAGATCCAAGTTTGTA |
| | | CAAAAAAGCAGGCTGCCACCATGGTGAG |
| | | CAAGGGCGAGGAGCTGTTCACCGGGGTG |
| | | GTGCCCATCCTGGTCGAGCTGGACGGCG |
| | | ACGTAAACGGCCACAAGTTCAGCGTGTC |
| | | CGGCGAGGGCGAGGGCGATGCCACCTAC |
| | | GGCAAGCTGACCCTGAAGTTCATCTGCA |
| | | CCACCGGCAAGCTGCCCGTGCCCTGGCC |
| | | CACCCTCGTGACCACCCTGACCTACGGC |
| | | GTGCAGTGCTTCAGCCGCTACCCCGACC |
| | | ACATGAAGCAGCACGACTTCTTCAAGTC |
| | | CGCCATGCCCGAAGGCTACGTCCAGGAG |
| | | CGCACCATCTTCTTCAAGGACGACGGCA |
| | | ACTACAAGACCCGCGCCGAGGTGAAGTT |
| | | CGAGGGCGACACCCTGGTGAACCGCATC |
| | | GAGCTGAAGGGCATCGACTTCAAGGAGG |
| | | ACGGCAACATCCTGGGGCACAAGCTGGA |
| | | GTACAACTACAACAGCCACAACGTCTAT |
| | | ATCATGGCCGACAAGCAGAAGAACGGCA |
| | | TCAAGGTGAACTTCAAGATCCGCCACAA |
| | | CATCGAGGACGGCAGCGTGCAGCTCGCC |
| | | GACCACTACCAGCAGAACACCCCCATCG |
| | | GCGACGGCCCCGTGCTGCTGCCCGACAA |
| | | CCACTACCTGAGCACCCAGTCCGCCCTG |
| | | AGCAAAGACCCCAACGAGAAGCGCGATC |
| | | ACATGGTCCTGCTGGAGTTCGTGACCGC |
| | | CGCCGGGATCACTCTCGGCATGGACGAG |
| | | CTGTACAAGTAAACCCAGCTTTCTTGTA |
| | | CAAAGTGGGAATTCCGATAATCAACCTC |
| | | TGGATTACAAAATTTGTGAAAGATTGAC |
| | | TGGTATTCTTAACTATGTTGCTCCTTTT |
| | | ACGCTATGTGGATACGCTGCTTTAATGC |
| | | CTTTGTATCATGCTATTGCTTCCCGTAT |
| | | GGCTTTCATTTTCTCCTCCTTGTATAAA |
| | | TCCTGGTTGCTGTCTCTTTATGAGGAGT |
| | | TGTGGCCCGTTGTCAGGCAACGTGGCGT |
| | | GGTGTGCACTGTGTTTGCTGACGCAACC |
| | | CCCACTGGTTGGGGCATTGCCACCACCT |
| | | GTCAGCTCCTTTCCGGGACTTTCGCTTT |
| | | CCCCCTCCCTATTGCCACGGCGGAACTC |
| | | ATCGCCGCCTGCCTTGCCCGCTGCTGGA |
| | | CAGGGGCTCGGCTGTTGGGCACTGACAA |
| | | TTCCGTGGTGTTGTCGGGGAAGCTGACG |
| | | TCCTTTCCATGGCTGCTCGCCTGTGTTG |
| | | CCACCTGGATTCTGCGCGGGACGTCCTT |
| | | CTGCTACGTCCCTTCGGCCCTCAATCCA |
| | | GCGGACCTTCCTTCCCGCGGCCTGCTGC |
| | | CGGCTCTGCGGCCTCTTCCGCGTCTTCG |
| | | CCTTCGCCCTCAGACGAGTCGGATCTCC |
| | | CTTTGGGCCGCCTCCCCGCATCGGGAAT |
| | | TCCTAGAGCTCGCTGATCAGCCTCGACT |
| | | GTGCCTTCTAGTTGCCAGCCATCTGTTG |
| | | TTTGCCCCTCCCCCGTGCCTTCCTTGAC |
| | | CCTGGAAGGTGCCACTCCCACTGTCCTT |
| | | TCCTAATAAAATGAGGAAATTGCATCGC |
| | | ATTGTCTGAGTAGGTGTCATTCTATTCT |
| | | GGGGGGTGGGGTGGGGCAGGACAGCAAG |
| | | GGGGAGGATTGGGAAGAGAATAGCAGGC |
| | | ATGCTGGGGAGGGCCGCAGGAACCCCTA |
| | | GTGATGGAGTTGGCCACTCCCTCTCTGC |
| | | GCGCTCGCTCGCTCACTGAGGCCGGGCG |
| | | ACCAAAGGTCGCCCGACGCCCGGGCTTT |
| | | GCCCGGGCGGCCTCAGTGAGCGAGCGAG |
| | | CGCGCAGCTGCCTGCAGGGGCGCCTGAT |
| | | GCGGTATTTTCTCCTTACGCATCTGTGC |
| | | GGTATTTCACACCGCATACGTCAAAGCA |
| | | ACCATAGTACGCGCCCTGTAGCGGCGCA |
| | | TTAAGCGCGGCGGGTGTGGTGGTTACGC |
| | | GCAGCGTGACCGCTACACTTGCCAGCGC |
| | | CTTAGCGCCCGCTCCTTTCGCTTTCTTC |
| | | CCTTCCTTTCTCGCCACGTTCGCCGGCT |
| | | TTCCCCGTCAAGCTCTAAATCGGGGGCT |
| | | CCCTTTAGGGTTCCGATTTAGTGCTTTA |
| | | CGGCACCTCGACCCCAAAAAACTTGATT |
| | | TGGGTGATGGTTCACGTAGTGGGCCATC |
| | | GCCCTGATAGACGGTTTTTCGCCCTTTG |
| | | ACGTTGGAGTCCACGTTCTTTAATAGTG |
| | | GACTCTTGTTCCAAACTGGAACAACACT |
| | | CAACTCTATCTCGGGCTATTCTTTTGAT |
| | | TTATAAGGGATTTTGCCGATTTCGGTCT |
| | | ATTGGTTAAAAAATGAGCTGATTTAACA |
| | | AAAATTTAACGCGAATTTTAACAAAATA |
| | | TTAACGTTTACAATTTTATGGTGCACTC |
| | | TCAGTACAATCTGCTCTGATGCCGCATA |
| | | GTTAAGCCAGCCCCGACACCCGCCAACA |
| | | CCCGCTGACGCGCCCTGACGGGCTTGTC |
| | | TGCTCCCGGCATCCGCTTACAGACAAGC |
| | | TGTGACCGTCTCCGGGAGCTGCATGTGT |
| | | CAGAGGTTTTCACCGTCATCACCGAAAC |
| | | GCGCGAGACGAAAGGGCCTCGTGATACG |
| | | CCTATTTTTATAGGTTAATGTCATGATA |
| | | ATAATGGTTTCTTAGACGTCAGGTGGCA |
| | | CTTTTCGGGGAAATGTGCGCGGAACCCC |
| | | TATTTGTTTATTTTTCTAAATACATTCA |
| | | AATATGTATCCGCTCATGAGACAATAAC |
| | | CCTGATAAATGCTTCAATAATATTGAAA |
| | | AAGGAAGAGTATGAGTATTCAACATTTC |
| | | CGTGTCGCCCTTATTCCCTTTTTTGCGG |
| | | CATTTTGCCTTCCTGTTTTTGCTCACCC |
| | | AGAAACGCTGGTGAAAGTAAAAGATGCT |
| | | GAAGATCAGTTGGGTGCACGAGTGGGTT |
| | | ACATCGAACTGGATCTCAACAGCGGTAA |
| | | GATCCTTGAGAGTTTTCGCCCCGAAGAA |
| | | CGTTTTCCAATGATGAGCACTTTTAAAG |
| | | TTCTGCTATGTGGCGCGGTATTATCCCG |
| | | TATTGACGCCGGGCAAGAGCAACTCGGT |
| | | CGCCGCATACACTATTCTCAGAATGACT |
| | | TGGTTGAGTACTCACCAGTCACAGAAAA |
| | | GCATCTTACGGATGGCATGACAGTAAGA |
| | | GAATTATGCAGTGCTGCCATAACCATGA |
| | | GTGATAACACTGCGGCCAACTTACTTCT |
| | | GACAACGATCGGAGGACCGAAGGAGCTA |
| | | ACCGCTTTTTTGCACAACATGGGGGATC |
| | | ATGTAACTCGCCTTGATCGTTGGGAACC |
| | | GGAGCTGAATGAAGCCATACCAAACGAC |
| | | GAGCGTGACACCACGATGCCTGTAGCAA |
| | | TGGCAACAACGTTGCGCAAACTATTAAC |
| | | TGGCGAACTACTTACTCTAGCTTCCCGG |
| | | CAACAATTAATAGACTGGATGGAGGCGG |
| | | ATAAAGTTGCAGGACCACTTCTGCGCTC |
| | | GGCCCTTCCGGCTGGCTGGTTTATTGCT |
| | | GATAAATCTGGAGCCGGTGAGCGTGGAA |
| | | GCCGCGGTATCATTGCAGCACTGGGGCC |
| | | AGATGGTAAGCCCTCCCGTATCGTAGTT |
| | | ATCTACACGACGGGGAGTCAGGCAACTA |
| | | TGGATGAACGAAATAGACAGATCGCTGA |
| | | GATAGGTGCCTCACTGATTAAGCATTGG |
| | | TAACTGTCAGACCAAGTTTACTCATATA |
| | | TACTTTAGATTGATTTAAAACTTCATTT |
| | | TTAATTTAAAAGGATCTAGGTGAAGATC |
| | | CTTTTTGATAATCTCATGACCAAAATCC |
| | | CTTAACGTGAGTTTTCGTTCCACTGAGC |
| | | GTCAGACCCCGTAGAAAAGATCAAAGGA |
| | | TCTTCTTGAGATCCTTTTTTTCTGCGCG |
| | | TAATCTGCTGCTTGCAAACAAAAAAACC |
| | | ACCGCTACCAGCGGTGGTTTGTTTGCCG |
| | | GATCAAGAGCTACCAACTCTTTTTCCGA |
| | | AGGTAACTGGCTTCAGCAGAGCGCAGAT |
| | | ACCAAATACTGTTCTTCTAGTGTAGCCG |
| | | TAGTTAGGCCACCACTTCAAGAACTCTG |
| | | TAGCACCGCCTACATACCTCGCTCTGCT |
| | | AATCCTGTTACCAGTGGCTGCTGCCAGT |
| | | GGCGATAAGTCGTGTCTTACCGGGTTGG |
| | | ACTCAAGACGATAGTTACCGGATAAGGC |
| | | GCAGCGGTCGGGCTGAACGGGGGGTTCG |
| | | TGCACACAGCCCAGCTTGGAGCGAACGA |
| | | CCTACACCGAACTGAGATACCTACAGCG |
| | | TGAGCTATGAGAAAGCGCCACGCTTCCC |
| | | GAAGGGAGAAAGGCGGACAGGTATCCGG |
| | | TAAGCGGCAGGGTCGGAACAGGAGAGCG |
| | | CACGAGGGAGCTTCCAGGGGGAAACGCC |
| | | TGGTATCTTTATAGTCCTGTCGGGTTTC |
| | | GCCACCTCTGACTTGAGCGTCGATTTTT |
| | | GTGATGCTCGTCAGGGGGGCGGAGCCTA |
| | | TGGAAAAACGCCAGCAACGCGGCCTTTT |
| | | TACGGTTCCTGGCCTTTTGCTGGCCTTT |
| | | TGCTCACATGTCCTGCAGGCAG |
| 2 | AAV2-CMV- | CTGCGCGCTCGCTCGCTCACTGAGGCCG |
| | mCherry | CCCGGGCAAAGCCCGGGCGTCGGGCGAC |
| | | CTTTGGTCGCCCGGCCTCAGTGAGCGAG |
| | | CGAGCGCGCAGAGAGGGAGTGGCCAACT |
| | | CCATCACTAGGGGTTCCTTCTAGACAAC |
| | | TTTGTATAGAAAAGTTGTAGTTATTAAT |
| | | AGTAATCAATTACGGGGTCATTAGTTCA |
| | | TAGCCCATATATGGAGTTCCGCGTTACA |
| | | TAACTTACGGTAAATGGCCCGCCTGGCT |
| | | GACCGCCCAACGACCCCCGCCCATTGAC |
| | | GTCAATAATGACGTATGTTCCCATAGTA |
| | | ACGCCAATAGGGACTTTCCATTGACGTC |
| | | AATGGGTGGAGTATTTACGGTAAACTGC |
| | | CCACTTGGCAGTACATCAAGTGTATCAT |
| | | ATGCCAAGTACGCCCCCTATTGACGTCA |
| | | ATGACGGTAAATGGCCCGCCTGGCATTA |
| | | TGCCCAGTACATGACCTTATGGGACTTT |
| | | CCTACTTGGCAGTACATCTACGTATTAG |
| | | TCATCGCTATTACCATGGTGATGCGGTT |
| | | TTGGCAGTACATCAATGGGCGTGGATAG |
| | | CGGTTTGACTCACGGGGATTTCCAAGTC |
| | | TCCACCCCATTGACGTCAATGGGAGTTT |
| | | GTTTTGGCACCAAAATCAACGGGACTTT |
| | | CCAAAATGTCGTAACAACTCCGCCCCAT |
| | | TGACGCAAATGGGCGGTAGGCGTGTACG |
| | | GTGGGAGGTCTATATAAGCAGAGCTGGT |
| | | TTAGTGAACCGTCAGATCCAAGTTTGTA |
| | | CAAAAAAGCAGGCTGCCACCATGGTGAG |
| | | CAAGGGCGAGGAGGATAACATGGCCATC |
| | | ATCAAGGAGTTCATGCGCTTCAAGGTGC |
| | | ACATGGAGGGCTCCGTGAACGGCCACGA |
| | | GTTCGAGATCGAGGGCGAGGGCGAGGGC |
| | | CGCCCCTACGAGGGCACCCAGACCGCCA |
| | | AGCTGAAGGTGACCAAGGGTGGCCCCCT |
| | | GCCCTTCGCCTGGGACATCCTGTCCCCT |
| | | CAGTTCATGTACGGCTCCAAGGCCTACG |
| | | TGAAGCACCCCGCCGACATCCCCGACTA |
| | | CTTGAAGCTGTCCTTCCCCGAGGGCTTC |
| | | AAGTGGGAGCGCGTGATGAACTTCGAGG |
| | | ACGGCGGCGTGGTGACCGTGACCCAGGA |
| | | CTCCTCCCTGCAGGACGGCGAGTTCATC |
| | | TACAAGGTGAAGCTGCGCGGCACCAACT |
| | | TCCCCTCCGACGGCCCCGTAATGCAGAA |
| | | GAAGACCATGGGCTGGGAGGCCTCCTCC |
| | | GAGCGGATGTACCCCGAGGACGGCGCCC |
| | | TGAAGGGCGAGATCAAGCAGAGGCTGAA |
| | | GCTGAAGGACGGCGGCCACTACGACGCT |
| | | GAGGTCAAGACCACCTACAAGGCCAAGA |
| | | AGCCCGTGCAGCTGCCCGGCGCCTACAA |
| | | CGTCAACATCAAGTTGGACATCACCTCC |
| | | CACAACGAGGACTACACCATCGTGGAAC |
| | | AGTACGAACGCGCCGAGGGCCGCCACTC |
| | | CACCGGCGGCATGGACGAGCTGTACAAG |
| | | TAAACCCAGCTTTCTTGTACAAAGTGGG |
| | | AATTCCGATAATCAACCTCTGGATTACA |
| | | AAATTTGTGAAAGATTGACTGGTATTCT |
| | | TAACTATGTTGCTCCTTTTACGCTATGT |
| | | GGATACGCTGCTTTAATGCCTTTGTATC |
| | | ATGCTATTGCTTCCCGTATGGCTTTCAT |
| | | TTTCTCCTCCTTGTATAAATCCTGGTTG |
| | | CTGTCTCTTTATGAGGAGTTGTGGCCCG |
| | | TTGTCAGGCAACGTGGCGTGGTGTGCAC |
| | | TGTGTTTGCTGACGCAACCCCCACTGGT |
| | | TGGGGCATTGCCACCACCTGTCAGCTCC |
| | | TTTCCGGGACTTTCGCTTTCCCCCTCCC |
| | | TATTGCCACGGCGGAACTCATCGCCGCC |
| | | TGCCTTGCCCGCTGCTGGACAGGGGCTC |
| | | GGCTGTTGGGCACTGACAATTCCGTGGT |
| | | GTTGTCGGGGAAGCTGACGTCCTTTCCA |
| | | TGGCTGCTCGCCTGTGTTGCCACCTGGA |
| | | TTCTGCGCGGGACGTCCTTCTGCTACGT |
| | | CCCTTCGGCCCTCAATCCAGCGGACCTT |
| | | CCTTCCCGCGGCCTGCTGCCGGCTCTGC |
| | | GGCCTCTTCCGCGTCTTCGCCTTCGCCC |
| | | TCAGACGAGTCGGATCTCCCTTTGGGCC |
| | | GCCTCCCCGCATCGGGAATTCCTAGAGC |
| | | TCGCTGATCAGCCTCGACTGTGCCTTCT |
| | | AGTTGCCAGCCATCTGTTGTTTGCCCCT |
| | | CCCCCGTGCCTTCCTTGACCCTGGAAGG |
| | | TGCCACTCCCACTGTCCTTTCCTAATAA |
| | | AATGAGGAAATTGCATCGCATTGTCTGA |
| | | GTAGGTGTCATTCTATTCTGGGGGGTGG |
| | | GGTGGGGCAGGACAGCAAGGGGGAGGAT |
| | | TGGGAAGAGAATAGCAGGCATGCTGGGG |
| | | AGGGCCGCAGGAACCCCTAGTGATGGAG |
| | | TTGGCCACTCCCTCTCTGCGCGCTCGCT |
| | | CGCTCACTGAGGCCGGGCGACCAAAGGT |
| | | CGCCCGACGCCCGGGCTTTGCCCGGGCG |
| | | GCCTCAGTGAGCGAGCGAGCGCGCAGCT |
| | | GCCTGCAGGGGCGCCTGATGCGGTATTT |
| | | TCTCCTTACGCATCTGTGCGGTATTTCA |
| | | CACCGCATACGTCAAAGCAACCATAGTA |
| | | CGCGCCCTGTAGCGGCGCATTAAGCGCG |
| | | GCGGGGGTGGTGGTTACGCGCAGCGTGA |
| | | CCGCTACACTTGCCAGCGCCTTAGCGCC |
| | | CGCTCCTTTCGCTTTCTTCCCTTCCTTT |
| | | CTCGCCACGTTCGCCGGCTTTCCCCGTC |
| | | AAGCTCTAAATCGGGGGCTCCCTTTAGG |
| | | GTTCCGATTTAGTGCTTTACGGCACCTC |
| | | GACCCCAAAAAACTTGATTTGGGTGATG |
| | | GTTCACGTAGTGGGCCATCGCCCTGATA |
| | | GACGGTTTTTCGCCCTTTGACGTTGGAG |
| | | TCCACGTTCTTTAATAGTGGACTCTTGT |
| | | TCCAAACTGGAACAACACTCAACTCTAT |
| | | CTCGGGCTATTCTTTTGATTTATAAGGG |
| | | ATTTTGCCGATTTCGGTCTATTGGTTAA |
| | | AAAATGAGCTGATTTAACAAAAATTTAA |
| | | CGCGAATTTTAACAAAATATTAACGTTT |
| | | ACAATTTTATGGTGCACTCTCAGTACAA |
| | | TCTGCTCTGATGCCGCATAGTTAAGCCA |
| | | GCCCCGACACCCGCCAACACCCGCTGAC |
| | | GCGCCCTGACGGGCTTGTCTGCTCCCGG |
| | | CATCCGCTTACAGACAAGCTGTGACCGT |
| | | CTCCGGGAGCTGCATGTGTCAGAGGTTT |
| | | TCACCGTCATCACCGAAACGCGCGAGAC |
| | | GAAAGGGCCTCGTGATACGCCTATTTTT |
| | | ATAGGTTAATGTCATGATAATAATGGTT |
| | | TCTTAGACGTCAGGTGGCACTTTTCGGG |
| | | GAAATGTGCGCGGAACCCCTATTTGTTT |
| | | ATTTTTCTAAATACATTCAAATATGTAT |
| | | CCGCTCATGAGACAATAACCCTGATAAA |
| | | TGCTTCAATAATATTGAAAAAGGAAGAG |
| | | TATGAGTATTCAACATTTCCGTGTCGCC |
| | | CTTATTCCCTTTTTTGCGGCATTTTGCC |
| | | TTCCTGTTTTTGCTCACCCAGAAACGCT |
| | | GGTGAAAGTAAAAGATGCTGAAGATCAG |
| | | TTGGGTGCACGAGTGGGTTACATCGAAC |
| | | TGGATCTCAACAGCGGTAAGATCCTTGA |
| | | GAGTTTTCGCCCCGAAGAACGTTTTCCA |
| | | ATGATGAGCACTTTTAAAGTTCTGCTAT |
| | | GTGGCGCGGTATTATCCCGTATTGACGC |
| | | CGGGCAAGAGCAACTCGGTCGCCGCATA |
| | | CACTATTCTCAGAATGACTTGGTTGAGT |
| | | ACTCACCAGTCACAGAAAAGCATCTTAC |
| | | GGATGGCATGACAGTAAGAGAATTATGC |
| | | AGTGCTGCCATAACCATGAGTGATAACA |
| | | CTGCGGCCAACTTACTTCTGACAACGAT |
| | | CGGAGGACCGAAGGAGCTAACCGCTTTT |
| | | TTGCACAACATGGGGGATCATGTAACTC |
| | | GCCTTGATCGTTGGGAACCGGAGCTGAA |
| | | TGAAGCCATACCAAACGACGAGCGTGAC |
| | | ACCACGATGCCTGTAGCAATGGCAACAA |
| | | CGTTGCGCAAACTATTAACTGGCGAACT |
| | | ACTTACTCTAGCTTCCCGGCAACAATTA |
| | | ATAGACTGGATGGAGGCGGATAAAGTTG |
| | | CAGGACCACTTCTGCGCTCGGCCCTTCC |
| | | GGCTGGCTGGTTTATTGCTGATAAATCT |
| | | GGAGCCGGTGAGCGTGGAAGCCGCGGTA |
| | | TCATTGCAGCACTGGGGCCAGATGGTAA |
| | | GCCCTCCCGTATCGTAGTTATCTACACG |
| | | ACGGGGAGTCAGGCAACTATGGATGAAC |
| | | GAAATAGACAGATCGCTGAGATAGGTGC |
| | | CTCACTGATTAAGCATTGGTAACTGTCA |
| | | GACCAAGTTTACTCATATATACTTTAGA |
| | | TTGATTTAAAACTTCATTTTTAATTTAA |
| | | AAGGATCTAGGTGAAGATCCTTTTTGAT |
| | | AATCTCATGACCAAAATCCCTTAACGTG |
| | | AGTTTTCGTTCCACTGAGCGTCAGACCC |
| | | CGTAGAAAAGATCAAAGGATCTTCTTGA |
| | | GATCCTTTTTTTCTGCGCGTAATCTGCT |
| | | GCTTGCAAACAAAAAAACCACCGCTACC |
| | | AGCGGTGGTTTGTTTGCCGGATCAAGAG |
| | | CTACCAACTCTTTTTCCGAAGGTAACTG |
| | | GCTTCAGCAGAGCGCAGATACCAAATAC |
| | | TGTTCTTCTAGTGTAGCCGTAGTTAGGC |
| | | CACCACTTCAAGAACTCTGTAGCACCGC |
| | | CTACATACCTCGCTCTGCTAATCCTGTT |
| | | ACCAGTGGCTGCTGCCAGTGGCGATAAG |
| | | TCGTGTCTTACCGGGTTGGACTCAAGAC |
| | | GATAGTTACCGGATAAGGCGCAGCGGTC |
| | | GGGCTGAACGGGGGGTTCGTGCACACAG |
| | | CCCAGCTTGGAGCGAACGACCTACACCG |
| | | AACTGAGATACCTACAGCGTGAGCTATG |
| | | AGAAAGCGCCACGCTTCCCGAAGGGAGA |
| | | AAGGCGGACAGGTATCCGGTAAGCGGCA |
| | | GGGTCGGAACAGGAGAGCGCACGAGGGA |
| | | GCTTCCAGGGGGAAACGCCTGGTATCTT |
| | | TATAGTCCTGTCGGGTTTCGCCACCTCT |
| | | GACTTGAGCGTCGATTTTTGTGATGCTC |
| | | GTCAGGGGGGCGGAGCCTATGGAAAAAC |
| | | GCCAGCAACGCGGCCTTTTTACGGTTCC |
| | | TGGCCTTTTGCTGGCCTTTTGCTCACAT |
| | | GTCCTGCAGGCAG |
| 3 | AAV2-CMV- | CCTGCAGGCAGCTGCGCGCTCGCTCGCT |
| | GDNF | CACTGAGGCCGCCCGGGCAAAGCCCGGG |
| | | CGTCGGGCGACCTTTGGTCGCCCGGCCT |
| | | CAGTGAGCGAGCGAGCGCGCAGAGAGGG |
| | | AGTGGCCAACTCCATCACTAGGGGTTCC |
| | | TTCTAGACAACTTTGTATAGAAAAGTTG |
| | | TAGTTATTAATAGTAATCAATTACGGGG |
| | | TCATTAGTTCATAGCCCATATATGGAGT |
| | | TCCGCGTTACATAACTTACGGTAAATGG |
| | | CCCGCCTGGCTGACCGCCCAACGACCCC |
| | | CGCCCATTGACGTCAATAATGACGTATG |
| | | TTCCCATAGTAACGCCAATAGGGACTTT |
| | | CCATTGACGTCAATGGGTGGAGTATTTA |
| | | CGGTAAACTGCCCACTTGGCAGTACATC |
| | | AAGTGTATCATATGCCAAGTACGCCCCC |
| | | TATTGACGTCAATGACGGTAAATGGCCC |
| | | GCCTGGCATTATGCCCAGTACATGACCT |
| | | TATGGGACTTTCCTACTTGGCAGTACAT |
| | | CTACGTATTAGTCATCGCTATTACCATG |
| | | GTGATGCGGTTTTGGCAGTACATCAATG |
| | | GGCGTGGATAGCGGTTTGACTCACGGGG |
| | | ATTTCCAAGTCTCCACCCCATTGACGTC |
| | | AATGGGAGTTTGTTTTGGCACCAAAATC |
| | | AACGGGACTTTCCAAAATGTCGTAACAA |
| | | CTCCGCCCCATTGACGCAAATGGGCGGT |
| | | AGGCGTGTACGGTGGGAGGTCTATATAA |
| | | GCAGAGCTGGTTTAGTGAACCGTCAGAT |
| | | CCAAGTTTGTACAAAAAAGCAGGCTGCC |
| | | ACCATGCAGTCTTTGCCTAACAGCAATG |
| | | GTGCCGCCGCCGGACGGGACTTTAAGAT |
| | | GAAGTTATGGGATGTCGTGGCTGTCTGC |
| | | CTGGTGCTGCTCCACACCGCGTCCGCCT |
| | | TCCCGCTGCCCGCCGGTAAGAGGCCTCC |
| | | CGAGGCGCCCGCCGAAGACCGCTCCCTC |
| | | GGCCGCCGCCGCGCGCCCTTCGCGCTGA |
| | | GCAGTGACTCAAATATGCCAGAGGATTA |
| | | TCCTGATCAGTTCGATGATGTCATGGAT |
| | | TTTATTCAAGCCACCATTAAAAGACTGA |
| | | AAAGGTCACCAGATAAACAAATGGCAGT |
| | | GCTTCCTAGAAGAGAGCGGAATCGGCAG |
| | | GCTGCAGCTGCCAACCCAGAGAATTCCA |
| | | GAGGAAAAGGTCGGAGAGGCCAGAGGGG |
| | | CAAAAACCGGGGTTGTGTCTTAACTGCA |
| | | ATACATTTAAATGTCACTGACTTGGGTC |
| | | TGGGCTATGAAACCAAGGAGGAACTGAT |
| | | TTTTAGGTACTGCAGCGGCTCTTGCGAT |
| | | GCAGCTGAGACAACGTACGACAAAATAT |
| | | TGAAAAACTTATCCAGAAATAGAAGGCT |
| | | GGTGAGTGACAAAGTAGGGCAGGCATGT |
| | | TGCAGACCCATCGCCTTTGATGATGACC |
| | | TGTCGTTTTTAGATGATAACCTGGTTTA |
| | | CCATATTCTAAGAAAGCATTCCGCTAAA |
| | | AGGTGTGGATGTATCTGAACCCAGCTTT |
| | | CTTGTACAAAGTGGGAATTCCTAGAGCT |
| | | CGCTGATCAGCCTCGACTGTGCCTTCTA |
| | | GTTGCCAGCCATCTGTTGTTTGCCCCTC |
| | | CCCCGTGCCTTCCTTGACCCTGGAAGGT |
| | | GCCACTCCCACTGTCCTTTCCTAATAAA |
| | | ATGAGGAAATTGCATCGCATTGTCTGAG |
| | | TAGGTGTCATTCTATTCTGGGGGGTGGG |
| | | GTGGGGCAGGACAGCAAGGGGGAGGATT |
| | | GGGAAGAGAATAGCAGGCATGCTGGGGA |
| | | GGGCCGCAGGAACCCCTAGTGATGGAGT |
| | | TGGCCACTCCCTCTCTGCGCGCTCGCTC |
| | | GCTCACTGAGGCCGGGCGACCAAAGGTC |
| | | GCCCGACGCCCGGGCTTTGCCCGGGCGG |
| | | CCTCAGTGAGCGAGCGAGCGCGCAGCTG |
| | | CCTGCAGGGGCGCCTGATGCGGTATTTT |
| | | CTCCTTACGCATCTGTGCGGTATTTCAC |
| | | ACCGCATACGTCAAAGCAACCATAGTAC |
| | | GCGCCCTGTAGCGGCGCATTAAGCGCGG |
| | | CGGGGGTGGTGGTTACGCGCAGCGTGAC |
| | | CGCTACACTTGCCAGCGCCTTAGCGCCC |
| | | GCTCCTTTCGCTTTCTTCCCTTCCTTTC |
| | | TCGCCACGTTCGCCGGCTTTCCCCGTCA |
| | | AGCTCTAAATCGGGGGCTCCCTTTAGGG |
| | | TTCCGATTTAGTGCTTTACGGCACCTCG |
| | | ACCCCAAAAAACTTGATTTGGGTGATGG |
| | | TTCACGTAGTGGGCCATCGCCCTGATAG |
| | | ACGGTTTTTCGCCCTTTGACGTTGGAGT |
| | | CCACGTTCTTTAATAGTGGACTCTTGTT |
| | | CCAAACTGGAACAACACTCAACTCTATC |
| | | TCGGGCTATTCTTTTGATTTATAAGGGA |
| | | TTTTGCCGATTTCGGTCTATTGGTTAAA |
| | | AAATGAGCTGATTTAACAAAAATTTAAC |
| | | GCGAATTTTAACAAAATATTAACGTTTA |
| | | CAATTTTATGGTGCACTCTCAGTACAAT |
| | | CTGCTCTGATGCCGCATAGTTAAGCCAG |
| | | CCCCGACACCCGCCAACACCCGCTGACG |
| | | CGCCCTGACGGGCTTGTCTGCTCCCGGC |
| | | ATCCGCTTACAGACAAGCTGTGACCGTC |
| | | TCCGGGAGCTGCATGTGTCAGAGGTTTT |
| | | CACCGTCATCACCGAAACGCGCGAGACG |
| | | AAAGGGCCTCGTGATACGCCTATTTTTA |
| | | TAGGTTAATGTCATGATAATAATGGTTT |
| | | CTTAGACGTCAGGTGGCACTTTTCGGGG |
| | | AAATGTGCGCGGAACCCCTATTTGTTTA |
| | | TTTTTCTAAATACATTCAAATATGTATC |
| | | CGCTCATGAGACAATAACCCTGATAAAT |
| | | GCTTCAATAATATTGAAAAAGGAAGAGT |
| | | ATGAGTATTCAACATTTCCGTGTCGCCC |
| | | TTATTCCCTTTTTTGCGGCATTTTGCCT |
| | | TCCTGTTTTTGCTCACCCAGAAACGCTG |
| | | GTGAAAGTAAAAGATGCTGAAGATCAGT |
| | | TGGGTGCACGAGTGGGTTACATCGAACT |
| | | GGATCTCAACAGCGGTAAGATCCTTGAG |
| | | AGTTTTCGCCCCGAAGAACGTTTTCCAA |
| | | TGATGAGCACTTTTAAAGTTCTGCTATG |
| | | TGGCGCGGTATTATCCCGTATTGACGCC |
| | | GGGCAAGAGCAACTCGGTCGCCGCATAC |
| | | ACTATTCTCAGAATGACTTGGTTGAGTA |
| | | CTCACCAGTCACAGAAAAGCATCTTACG |
| | | GATGGCATGACAGTAAGAGAATTATGCA |
| | | GTGCTGCCATAACCATGAGTGATAACAC |
| | | TGCGGCCAACTTACTTCTGACAACGATC |
| | | GGAGGACCGAAGGAGCTAACCGCTTTTT |
| | | TGCACAACATGGGGGATCATGTAACTCG |
| | | CCTTGATCGTTGGGAACCGGAGCTGAAT |
| | | GAAGCCATACCAAACGACGAGCGTGACA |
| | | CCACGATGCCTGTAGCAATGGCAACAAC |
| | | GTTGCGCAAACTATTAACTGGCGAACTA |
| | | CTTACTCTAGCTTCCCGGCAACAATTAA |
| | | TAGACTGGATGGAGGCGGATAAAGTTGC |
| | | AGGACCACTTCTGCGCTCGGCCCTTCCG |
| | | GCTGGCTGGTTTATTGCTGATAAATCTG |
| | | GAGCCGGTGAGCGTGGAAGCCGCGGTAT |
| | | CATTGCAGCACTGGGGCCAGATGGTAAG |
| | | CCCTCCCGTATCGTAGTTATCTACACGA |
| | | CGGGGAGTCAGGCAACTATGGATGAACG |
| | | AAATAGACAGATCGCTGAGATAGGTGCC |
| | | TCACTGATTAAGCATTGGTAACTGTCAG |
| | | ACCAAGTTTACTCATATATACTTTAGAT |
| | | TGATTTAAAACTTCATTTTTAATTTAAA |
| | | AGGATCTAGGTGAAGATCCTTTTTGATA |
| | | ATCTCATGACCAAAATCCCTTAACGTGA |
| | | GTTTTCGTTCCACTGAGCGTCAGACCCC |
| | | GTAGAAAAGATCAAAGGATCTTCTTGAG |
| | | ATCCTTTTTTTCTGCGCGTAATCTGCTG |
| | | CTTGCAAACAAAAAAACCACCGCTACCA |
| | | GCGGTGGTTTGTTTGCCGGATCAAGAGC |
| | | TACCAACTCTTTTTCCGAAGGTAACTGG |
| | | CTTCAGCAGAGCGCAGATACCAAATACT |
| | | GTTCTTCTAGTGTAGCCGTAGTTAGGCC |
| | | ACCACTTCAAGAACTCTGTAGCACCGCC |
| | | TACATACCTCGCTCTGCTAATCCTGTTA |
| | | CCAGTGGCTGCTGCCAGTGGCGATAAGT |
| | | CGTGTCTTACCGGGTTGGACTCAAGACG |
| | | ATAGTTACCGGATAAGGCGCAGCGGTCG |
| | | GGCTGAACGGGGGGTTCGTGCACACAGC |
| | | CCAGCTTGGAGCGAACGACCTACACCGA |
| | | ACTGAGATACCTACAGCGTGAGCTATGA |
| | | GAAAGCGCCACGCTTCCCGAAGGGAGAA |
| | | AGGCGGACAGGTATCCGGTAAGCGGCAG |
| | | GGTCGGAACAGGAGAGCGCACGAGGGAG |
| | | CTTCCAGGGGGAAACGCCTGGTATCTTT |
| | | ATAGTCCTGTCGGGTTTCGCCACCTCTG |
| | | ACTTGAGCGTCGATTTTTGTGATGCTCG |
| | | TCAGGGGGGCGGAGCCTATGGAAAAACG |
| | | CCAGCAACGCGGCCTTTTTACGGTTCCT |
| | | GGCCTTTTGCTGGCCTTTTGCTCACATG |
| | | T |
| 4 | AAV2-TRE- | CCTGCAGGCAGCTGCGCGCTCGCTCGCT |
| | GDNF | CACTGAGGCCGCCCGGGCAAAGCCCGGG |
| | | CGTCGGGCGACCTTTGGTCGCCCGGCCT |
| | | CAGTGAGCGAGCGAGCGCGCAGAGAGGG |
| | | AGTGGCCAACTCCATCACTAGGGGTTCC |
| | | TATCGATCAACTTTGTATAGAAAAGTTG |
| | | GGTACCGAGCTCGACTTTCACTTTTCTC |
| | | TATCACTGATAGGGAGTGGTAAACTCGA |
| | | CTTTCACTTTTCTCTATCACTGATAGGG |
| | | AGTGGTAAACTCGACTTTCACTTTTCTC |
| | | TATCACTGATAGGGAGTGGTAAACTCGA |
| | | CTTTCACTTTTCTCTATCACTGATAGGG |
| | | AGTGGTAAACTCGACTTTCACTTTTCTC |
| | | TATCACTGATAGGGAGTGGTAAACTCGA |
| | | CTTTCACTTTTCTCTATCACTGATAGGG |
| | | AGTGGTAAACTCGACTTTCACTTTTCTC |
| | | TATCACTGATAGGGAGTGGTAAACTCGA |
| | | CCTATATAAGCAGAGCTCGTTTAGTGAA |
| | | CCGTCAGATCGCCTGGAGACGCCATCCA |
| | | CGCTGTTTTGACCTCCATAGAAGACACC |
| | | GGGACCGATCCAGCCTCCGCGGCCCCGA |
| | | ATTGCAAGTTTGTACAAAAAAGCAGGCT |
| | | GCCACCATGCAGTCTTTGCCTAACAGCA |
| | | ATGGTGCCGCCGCCGGACGGGACTTTAA |
| | | GATGAAGTTATGGGATGTCGTGGCTGTC |
| | | TGCCTGGTGCTGCTCCACACCGCGTCCG |
| | | CCTTCCCGCTGCCCGCCGGTAAGAGGCC |
| | | TCCCGAGGCGCCCGCCGAAGACCGCTCC |
| | | CTCGGCCGCCGCCGCGCGCCCTTCGCGC |
| | | TGAGCAGTGACTCAAATATGCCAGAGGA |
| | | TTATCCTGATCAGTTCGATGATGTCATG |
| | | GATTTTATTCAAGCCACCATTAAAAGAC |
| | | TGAAAAGGTCACCAGATAAACAAATGGC |
| | | AGTGCTTCCTAGAAGAGAGCGGAATCGG |
| | | CAGGCTGCAGCTGCCAACCCAGAGAATT |
| | | CCAGAGGAAAAGGTCGGAGAGGCCAGAG |
| | | GGGCAAAAACCGGGGTTGTGTCTTAACT |
| | | GCAATACATTTAAATGTCACTGACTTGG |
| | | GTCTGGGCTATGAAACCAAGGAGGAACT |
| | | GATTTTTAGGTACTGCAGCGGCTCTTGC |
| | | GATGCAGCTGAGACAACGTACGACAAAA |
| | | TATTGAAAAACTTATCCAGAAATAGAAG |
| | | GCTGGTGAGTGACAAAGTAGGGCAGGCA |
| | | TGTTGCAGACCCATCGCCTTTGATGATG |
| | | ACCTGTCGTTTTTAGATGATAACCTGGT |
| | | TTACCATATTCTAAGAAAGCATTCCGCT |
| | | AAAAGGTGTGGATGTATCTGAACCCAGC |
| | | TTTCTTGTACAAAGTGGTGATGGCCGGC |
| | | CGCTTCGAGCAGACATGATAAGATACAT |
| | | TGATGAGTTTGGACAAACCACAACTAGA |
| | | ATGCAGTGAAAAAAATGCTTTATTTGTG |
| | | AAATTTGTGATGCTATTGCTTTATTTGT |
| | | AACCATTATAAGCTGCAATAAACAAGTT |
| | | AACAACAACAATTGCATTCATTTTATGT |
| | | TTCAGGTTCAGGGGGAGGTGTGGGAGGT |
| | | TTTTTAAAGCAAGTAAAACCTCTACAAA |
| | | TGTGGTAATCGATCCATAGAGCCCACCG |
| | | CATCCCCAGCATGCCTGCTATTGTCTTC |
| | | CCAATCCTCCCCCTTGCTGTCCTGCCCC |
| | | ACCCCACCCCCCAGAATAGAATGACACC |
| | | TACTCAGACAATGCGATGCAATTTCCTC |
| | | ATTTTATTAGGAAAGGACAGTGGGAGTG |
| | | GCACCTTCCAGGGTCAAGGAAGGCACGG |
| | | GGGAGGGGCAAACAACAGATGGCTGGCA |
| | | ACTAGAAGGCACAGTTACCCGGGGAGCA |
| | | TGTCAAGGTCAAAATCGTCAAGAGCGTC |
| | | AGCAGGCAGCATATCAAGGTCAAAGTCG |
| | | TCAAGGGCATCGGCTGGGAGCATGTCTA |
| | | AGTCAAAATCGTCAAGGGCGTCGGCCGG |
| | | CCCGCCGCTTTCGCACTTTAGCTGTTTC |
| | | TCCAGGCCACATATGATTAGTTCCAGGC |
| | | CGAAAAGGAAGGCAGGTTCGGCTCCCTG |
| | | CCGGTCGAACAGCTCAATTGCTTGTCTC |
| | | AGAAGTGGGGGCATAGAATCGGTGGTAG |
| | | GTGTCTCTCTTTCCTCTTTTGCTACTTG |
| | | ATGCTCCTGTTCCTCCAATACGCAGCCC |
| | | AGTGTAAAGTGGCCCACGGCGGACAGAG |
| | | CGTACAGTGCGTTCTCCAGGGAGAAGCC |
| | | TTGCTGACACAGGAACGCGAGCTGATTT |
| | | TCCAGGGTTTCGTACTGTTTCTCTGTTG |
| | | GGCGGGTGCCGAGATGCACTTTAGCCCC |
| | | GTCGCGATGTGAGAGGAGAGCACAGCGG |
| | | AATGACTTGGCGTTGTTCCGCAGAAAGT |
| | | CTTGCCATGACTCGCCTTCCAGGGGGCA |
| | | GAAGTGGGTATGATGCCTGTCCAGCATC |
| | | TCGATTGGCAGGGCATCGAGCAGGGCCC |
| | | GCTTGTTCTTCACGTGCCAGTACAGGGT |
| | | AGGCTGCTCAACTCCCAGCTTTTGAGCG |
| | | AGTTTCCTTGTCGTCAGGCCTTCGATAC |
| | | CGACTCCATTGAGTAATTCCAGAGCGCC |
| | | GTTTATGACTTTGCTCTTGTCCAGTCTA |
| | | GACATGGGGCCGGGATTTTCCTCCACGT |
| | | CCCCGCATGTTAGAAGACTTCCCCTGCC |
| | | CTCTCCGGAGCCCCAGGGATCCTCTCCT |
| | | TGCTGCAACAGGGAGATCACCTTTGGTT |
| | | TGGTAAACAGGAATCCTGCCATGGAGGC |
| | | CAGGTTGCTGTAATTCTCCAGCATCACC |
| | | TCACGGTACAGGCTTCTCTGAGACAGAT |
| | | CCAGCTTCTTCCACTCGTCCCGAGTAAA |
| | | GAGCACAGCCACATCTTCAAATGTCACT |
| | | GACACTGCTAGCTTTCTCTTCTTTTTTG |
| | | GTTTATTACCATCCTCAATGGGTGTATG |
| | | CTTTTTTTCATCTAACTGTGCAGAAAAT |
| | | CCAAATATTAATGATTTCAGCCCGAAAT |
| | | GGAAGGCCATTTCAGCAGAGGTTCTCGC |
| | | CTGAATATTAAATGCCTCTTGTAATAAT |
| | | GGTGGTGCAGCGTCTATCACATGATTAT |
| | | TTTCTATCTGGTTTGTTGCTTGCTCCTC |
| | | TAATACTGCACCCAACGTAAAATGGCTG |
| | | ATAGATTGCAGAATGAAAAGAGCCTCCT |
| | | CGACCGAAAACCCTGCATCGCATAGACA |
| | | GCGTAGTTGCGCCTCTGCTTGTTCAAAC |
| | | TGGGGGGGCGTAGGAGAGGTCCCTATAT |
| | | GCAATCGGGCTCCATCACGATGGACCAG |
| | | TAATGCTTTACGGAAACTCAGAGCATTT |
| | | TCCTGGAGAAACTGCTGCCAACTCTCAG |
| | | TCGGTAACGGTGCTGAACGGGTGTGATG |
| | | CTTCGCCAGTATTGCCTCTGAAAGCATG |
| | | TTCATAAGAGTCTGCTTGTTGCGCACGT |
| | | GCCAATACAATGTAGGCTGCTCTACACC |
| | | TAGCTTCTGGGCGAGTTTACGGGTTGTT |
| | | AAACCTTCGATTCCGACCTCATTAAGCA |
| | | GCTCTAATGCGCTGTTAATCACTTTACT |
| | | TTTATCTAATCTAGACATGAATTCCCAA |
| | | CCTGAAAAAAAGTGATTTCAGGCAGGTG |
| | | CTCCAGGTAATTAAACATTAATACCCCA |
| | | CCAACCAACCATCCCTTAAACCCTTACC |
| | | TCTTGCTCAGCTAATTACAGCCCGGAGG |
| | | AGAAGGGCCGTCCCGCCCGCTCACCTGT |
| | | GGGAGTAACGCGGTCAGTCAGAGCCGGG |
| | | GGGGGCGGCGCGAGGCGGCGGCGGAGCG |
| | | GGGCACGGGGCGAAGGCAGCGCGCAGCG |
| | | ACTCCCGCCCGCCGCGCGCTTCGCTTTT |
| | | TATAGGGCCGCCGCCGCCGCCGCCTCGC |
| | | CATAAAAGGAAACTTTCGGAGCGCGCCG |
| | | CTCTGATTGGCTGCCGCCGCACCTCTCC |
| | | GCCTCGCCCCGCCCCGCCCCTCGCCCCG |
| | | CCCCGCCCCGCCTGGCGCGCGCCCCCCC |
| | | CCCCCCCCCGCCCCCATCGCTGCACAAA |
| | | ATAATTAAAAAATAAATAAATACAAAAT |
| | | TGGGGGTGGGGAGGGGGGGGAGATGGGG |
| | | AGAGTGAAGCAGAACGTGGGGCTCACCT |
| | | CGACCATGGTAATAGCGATGACTAATAC |
| | | GTAGATGTACTGCCAAGTAGGAAAGTCC |
| | | CATAAGGTCATGTACTGGGCACAATGCC |
| | | AGGCGGGCCATTTACCGTCATTGACGTC |
| | | AATAGGGGGCGTACTTGGCATATGATAC |
| | | ACTTGATGTACTGCCAAGTGGGCAGTTT |
| | | ACCGTAAATACTCCACCCATTGACGTCA |
| | | ATGGAAAGTCCCTATTGGCGTTACTATT |
| | | GACGTCAATGGGGGGGGTCGTTGGGCGG |
| | | TCAGCCAGGCGGGCCATTTACCGTAAGT |
| | | TATGTAACGAGATCTAGGAACCCCTAGT |
| | | GATGGAGTTGGCCACTCCCTCTCTGCGC |
| | | GCTCGCTCGCTCACTGAGGCCGGGCGAC |
| | | CAAAGGTCGCCCGACGCCCGGGCTTTGC |
| | | CCGGGCGGCCTCAGTGAGCGAGCGAGCG |
| | | CGCAGCTGCCTGCAGGCAGCTTGGCACT |
| | | GGCCGTCGTTTTACAACGTCGTGACTGG |
| | | GAAAACCCTGGCGTTACCCAACTTAATC |
| | | GCCTTGCAGCACATCCCCCTTTCGCCAG |
| | | CTGGCGTAATAGCGAAGAGGCCCGCACC |
| | | GATCGCCCTTCCCAACAGTTGCGCAGCC |
| | | TGAATGGCGAATGGCGCCTGATGCGGTA |
| | | TTTTCTCCTTACGCATCTGTGCGGTATT |
| | | TCACACCGCATACGTCAAAGCAACCATA |
| | | GTACGCGCCCTGTAGCGGCGCATTAAGC |
| | | GCGGCGGGTGTGGTGGTTACGCGCAGCG |
| | | TGACCGCTACACTTGCCAGCGCCCTAGC |
| | | GCCCGCTCCTTTCGCTTTCTTCCCTTCC |
| | | TTTCTCGCCACGTTCGCCGGCTTTCCCC |
| | | GTCAAGCTCTAAATCGGGGGCTCCCTTT |
| | | AGGGTTCCGATTTAGTGCTTTACGGCAC |
| | | CTCGACCCCAAAAAACTTGATTTGGGTG |
| | | ATGGTTCACGTAGTGGGCCATCGCCCTG |
| | | ATAGACGGTTTTTCGCCCTTTGACGTTG |
| | | GAGTCCACGTTCTTTAATAGTGGACTCT |
| | | TGTTCCAAACTGGAACAACACTCAACCC |
| | | TATCTCGGGCTATTCTTTTGATTTATAA |
| | | GGGATTTTGCCGATTTCGGCCTATTGGT |
| | | TAAAAAATGAGCTGATTTAACAAAAATT |
| | | TAACGCGAATTTTAACAAAATATTAACG |
| | | TTTACAATTTTATGGTGCACTCTCAGTA |
| | | CAATCTGCTCTGATGCCGCATAGTTAAG |
| | | CCAGCCCCGACACCCGCCAACACCCGCT |
| | | GACGCGCCCTGACGGGCTTGTCTGCTCC |
| | | CGGCATCCGCTTACAGACAAGCTGTGAC |
| | | CGTCTCCGGGAGCTGCATGTGTCAGAGG |
| | | TTTTCACCGTCATCACCGAAACGCGCGA |
| | | GACGAAAGGGCCTCGTGATACGCCTATT |
| | | TTTATAGGTTAATGTCATGATAATAATG |
| | | GTTTCTTAGACGTCAGGTGGCACTTTTC |
| | | GGGGAAATGTGCGCGGAACCCCTATTTG |
| | | TTTATTTTTCTAAATACATTCAAATATG |
| | | TATCCGCTCATGAGACAATAACCCTGAT |
| | | AAATGCTTCAATAATATTGAAAAAGGAA |
| | | GAGTATGAGTATTCAACATTTCCGTGTC |
| | | GCCCTTATTCCCTTTTTTGCGGCATTTT |
| | | GCCTTCCTGTTTTTGCTCACCCAGAAAC |
| | | GCTGGTGAAAGTAAAAGATGCTGAAGAT |
| | | CAGTTGGGTGCACGAGTGGGTTACATCG |
| | | AACTGGATCTCAACAGCGGTAAGATCCT |
| | | TGAGAGTTTTCGCCCCGAAGAACGTTTT |
| | | CCAATGATGAGCACTTTTAAAGTTCTGC |
| | | TATGTGGCGCGGTATTATCCCGTATTGA |
| | | CGCCGGGCAAGAGCAACTCGGTCGCCGC |
| | | ATACACTATTCTCAGAATGACTTGGTTG |
| | | AGTACTCACCAGTCACAGAAAAGCATCT |
| | | TACGGATGGCATGACAGTAAGAGAATTA |
| | | TGCAGTGCTGCCATAACCATGAGTGATA |
| | | ACACTGCGGCCAACTTACTTCTGACAAC |
| | | GATCGGAGGACCGAAGGAGCTAACCGCT |
| | | TTTTTGCACAACATGGGGGATCATGTAA |
| | | CTCGCCTTGATCGTTGGGAACCGGAGCT |
| | | GAATGAAGCCATACCAAACGACGAGCGT |
| | | GACACCACGATGCCTGTAGCAATGGCAA |
| | | CAACGTTGCGCAAACTATTAACTGGCGA |
| | | ACTACTTACTCTAGCTTCCCGGCAACAA |
| | | TTAATAGACTGGATGGAGGCGGATAAAG |
| | | TTGCAGGACCACTTCTGCGCTCGGCCCT |
| | | TCCGGCTGGCTGGTTTATTGCTGATAAA |
| | | TCTGGAGCCGGTGAGCGTGGGTCTCGCG |
| | | GTATCATTGCAGCACTGGGGCCAGATGG |
| | | TAAGCCCTCCCGTATCGTAGTTATCTAC |
| | | ACGACGGGGAGTCAGGCAACTATGGATG |
| | | AACGAAATAGACAGATCGCTGAGATAGG |
| | | TGCCTCACTGATTAAGCATTGGTAACTG |
| | | TCAGACCAAGTTTACTCATATATACTTT |
| | | AGATTGATTTAAAACTTCATTTTTAATT |
| | | TAAAAGGATCTAGGTGAAGATCCTTTTT |
| | | GATAATCTCATGACCAAAATCCCTTAAC |
| | | GTGAGTTTTCGTTCCACTGAGCGTCAGA |
| | | CCCCGTAGAAAAGATCAAAGGATCTTCT |
| | | TGAGATCCTTTTTTTCTGCGCGTAATCT |
| | | GCTGCTTGCAAACAAAAAAACCACCGCT |
| | | ACCAGCGGTGGTTTGTTTGCCGGATCAA |
| | | GAGCTACCAACTCTTTTTCCGAAGGTAA |
| | | CTGGCTTCAGCAGAGCGCAGATACCAAA |
| | | TACTGTTCTTCTAGTGTAGCCGTAGTTA |
| | | GGCCACCACTTCAAGAACTCTGTAGCAC |
| | | CGCCTACATACCTCGCTCTGCTAATCCT |
| | | GTTACCAGTGGCTGCTGCCAGTGGCGAT |
| | | AAGTCGTGTCTTACCGGGTTGGACTCAA |
| | | GACGATAGTTACCGGATAAGGCGCAGCG |
| | | GTCGGGCTGAACGGGGGGTTCGTGCACA |
| | | CAGCCCAGCTTGGAGCGAACGACCTACA |
| | | CCGAACTGAGATACCTACAGCGTGAGCT |
| | | ATGAGAAAGCGCCACGCTTCCCGAAGGG |
| | | AGAAAGGCGGACAGGTATCCGGTAAGCG |
| | | GCAGGGTCGGAACAGGAGAGCGCACGAG |
| | | GGAGCTTCCAGGGGGAAACGCCTGGTAT |
| | | CTTTATAGTCCTGTCGGGTTTCGCCACC |
| | | TCTGACTTGAGCGTCGATTTTTGTGATG |
| | | CTCGTCAGGGGGGCGGAGCCTATGGAAA |
| | | AACGCCAGCAACGCGGCCTTTTTACGGT |
| | | TCCTGGCCTTTTGCTGGCCTTTTGCTCA |
| | | CATGTTCTTTCCTGCGTTATCCCCTGAT |
| | | TCTGTGGATAACCGTATTACCGCCTTTG |
| | | AGTGAGCTGATACCGCTCGCCGCAGCCG |
| | | AACGACCGAGCGCAGCGAGTCAGTGAGC |
| | | GAGGAAGCGGAAGAGCGCCCAATACGCA |
| | | AACCGCCTCTCCCCGCGCGTTGGCCGAT |
| | | TCATTAATGCAGCTGGCACGACAGGTTT |
| | | CCCGACTGGAAAGCGGGCAGTGAGCGCA |
| | | ACGCAATTAATGTGAGTTAGCTCACTCA |
| | | TTAGGCACCCCAGGCTTTACACTTTATG |
| | | CTTCCGGCTCGTATGTTGTGTGGAATTG |
| | | TGAGCGGATAACAATTTCACACAGGAAA |
| | | CAGCTATGACCATGATTACGAATTG |
| 5 | Human GDNF | ATGCAGTCTTTGCCTAACAGCAATGGTG |
| | | CCGCCGCCGGACGGGACTTTAAGATGAA |
| | | GTTATGGGATGTCGTGGCTGTCTGCCTG |
| | | GTGCTGCTCCACACCGCGTCCGCCTTCC |
| | | CGCTGCCCGCCGGTAAGAGGCCTCCCGA |
| | | GGCGCCCGCCGAAGACCGCTCCCTCGGC |
| | | CGCCGCCGCGCGCCCTTCGCGCTGAGCA |
| | | GTGACTCAAATATGCCAGAGGATTATCC |
| | | TGATCAGTTCGATGATGTCATGGATTTT |
| | | ATTCAAGCCACCATTAAAAGACTGAAAA |
| | | GGTCACCAGATAAACAAATGGCAGTGCT |
| | | TCCTAGAAGAGAGCGGAATCGGCAGGCT |
| | | GCAGCTGCCAACCCAGAGAATTCCAGAG |
| | | GAAAAGGTCGGAGAGGCCAGAGGGGCAA |
| | | AAACCGGGGTTGTGTCTTAACTGCAATA |
| | | CATTTAAATGTCACTGACTTGGGTCTGG |
| | | GCTATGAAACCAAGGAGGAACTGATTTT |
| | | TAGGTACTGCAGCGGCTCTTGCGATGCA |
| | | GCTGAGACAACGTACGACAAAATATTGA |
| | | AAAACTTATCCAGAAATAGAAGGCTGGT |
| | | GAGTGACAAAGTAGGGCAGGCATGTTGC |
| | | AGACCCATCGCCTTTGATGATGACCTGT |
| | | CGTTTTTAGATGATAACCTGGTTTACCA |
| | | TATTCTAAGAAAGCATTCCGCTAAAAGG |
| | | TGTGGATGTATCTGA |
| 6 | Human GDNF | MQSLPNSNGAAAGRDFKMKLWDVVAVCL |
| | amino acid | VLLHTASAFPLPAGKRPPEAPAEDRSLG |
| | sequence | RRRAPFALSSDSNMPEDYPDQFDDVMDF |
| | | IQATIKRLKRSPDKQMAVLPRRERNRQA |
| | | AAANPENSRGKGRRGQRGKNRGCVLTAI |
| | | HLNVTDLGLGYETKEELIFRYCSGSCDA |
| | | AETTYDKILKNLSRNRRLVSDKVGQACC |
| | | RPIAFDDDLSFLDDNLVYHILRKHSAKR |
| | | CGCI |

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Introduction

Previously, developed stretch-grown tissue engineered nerve grafts (TENGs) have been developed that facilitate regeneration following segmental nerve repair. See, for instance, Pfister, B. J., et al., J Neurosci. 2004; 24:7978-7983; and Huang, J. H., et al., Tissue Eng Part A. 2009; 15:1677-1685. Such a technique generally uses two populations of dorsal root ganglia (DRG) neurons on the proximal and distal ends, with parallel axon tracts/fascicles stretch-grown to span the two ends to achieve the desired length (see FIG. 2). TENGs have been validated in a 1-cm rat sciatic nerve and 5-cm pig hindlimb nerve transection models to effectively bridge and facilitate regeneration, performing equivalent to autografts. See, for instance, Katiyar, K. S., et al., Front Bioeng Biotechnol. 2020; 8:492.

As described herein, in a breakthrough that expands existing TENG strategies for neural repair, novel reprogrammed TENGs expressing neurogenic growth factor with temporal and spatial control using adeno-associated virus (AAV) gene transfer technology (AAV-TENGs) were developed. Notably, the differential spatial distribution of transgene expression for TENGs that can establish a gradient of neurotrophic growth factors was demonstrated. In addition, external temporal control of transgene expression was demonstrated using inducible tetracycline response element (TRE) promoter technology and doxycycline, a well-tolerated antibiotic in use clinically. The findings described herein demonstrated AAV-TENGs as a promising technology, not only to enhance regenerative strategies, but also for investigation of the effects of different growth factors in spatially and temporally controlled manner following PNI's.

### Example 1: Spatial Control of Transgene Expression from Novel Neural Scaffolds

Introduction: In this study, adeno associated viral vectors (AAV vectors) were used to drive TENG expression of neurotrophic factors under temporal and spatial control. In order to demonstrate expression control, TENGs were fabricated from embryonic rat dorsal root ganglia (DRG) transduced to express reporter or therapeutic genes. Importantly, proof-of-concept of effective DRG transduction and DRG expression of transgenes was shown. Ultimately, TENGs spanning up to 5 cm in length were designed and fabricated with preferential expression of different transgenes at different ends of the graft.

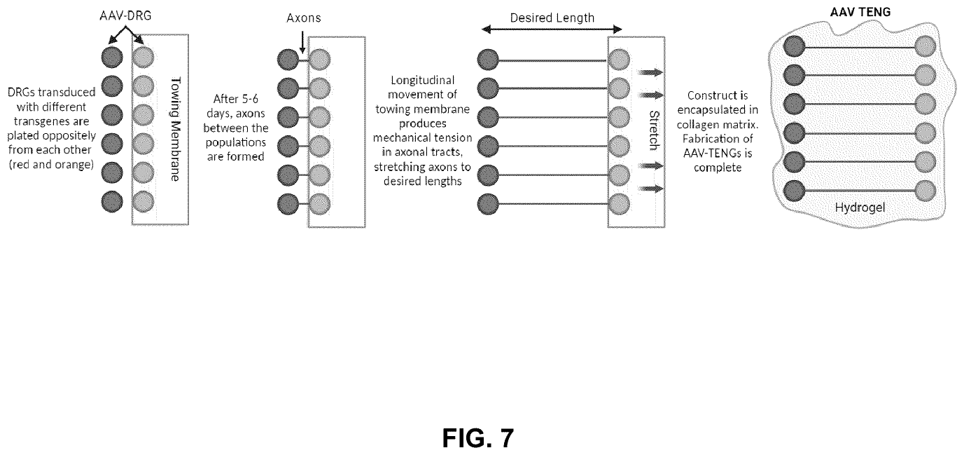

Methods: Dorsal root ganglia (DRG) were harvested from E16 rat embryos. Populations of DRG neurons were transduced with either AAV2-CMV-GFP, AAV2-CMV-mCherry, or AAV2-CMV-GDNF at 4° C. overnight, separately. DRGs expressing GFP were placed at left side of the plate, and DRGs expressing the mCherry or GDNF were placed at the right side of the plate. The two discrete populations, expressing different genes, were plated 1-2 mm apart in mechanobioreactors and maintained under 5% CO2 at 37° C. for 5 days. Mechanical tension was applied over 21 days, resulting in 5 cm long axon tracts. Shorter lengths were attained by stretching for less time. TENGs were encapsulated in collagen at the end. TENGs were imaged, cut in half, and placed in media, which was collected after 1 and 3 days. A general schematic representation of the generation of AAV TENGS is presented in FIG. 7.

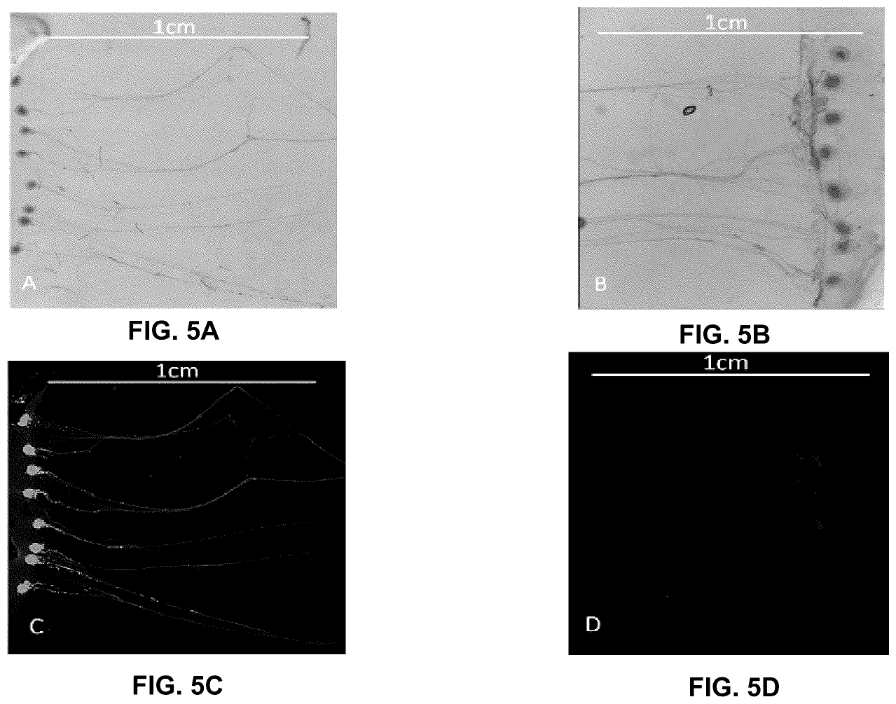

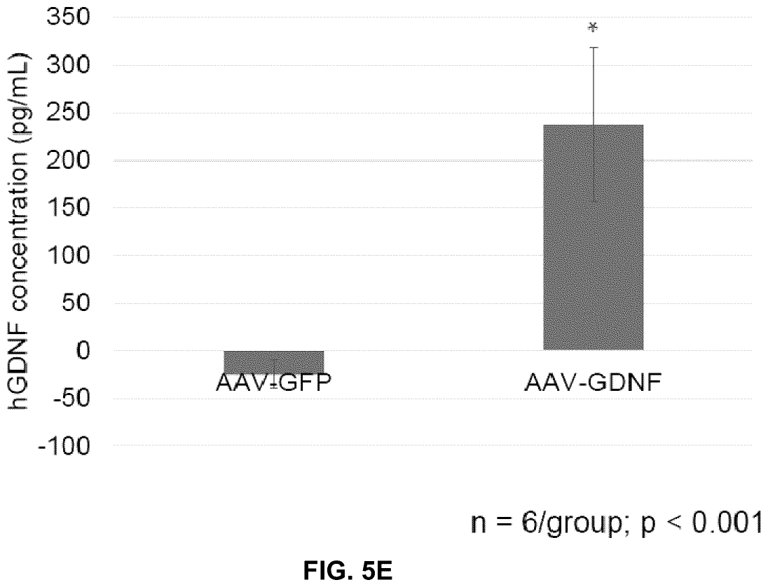

Results: Robust bright green or red expression was observed at each end of the AAV2-transduced DRG cell bodies as well as some lower signal along the axon tracts (FIG. 4A-FIG. 4C, FIG. 5C). Referring now to FIG. 4A-FIG. 4C, FIG. 4A-FIG. 4C present images of the differential expression of GFP and mCherry at each end of a 5 cm TENG. FIG. 4A presents an image of the 5 cm long TENG expressing GFP (left) and mCherry (right) at each of the respective ends. FIG. 4B presents an image the left side of the TENG transduced with AAV2-GFP. The left side of the TENG robustly expressed green axonal tracts. FIG. 4C presents an image of the right side of TENG DRGs transduced with AAV2-mCherry. The right side of the TENG expressed red axonal tracts and gradients of green axonal tract.

Referring now to FIG. 5A-FIG. 5E, FIG. 5A-FIG. 5E present images and a graphical representation of results related to the differential expression of GFP and GDNF at each end of a 5 cm TENG. FIG. 5A presents a brightfield image of the GFP side of the 5 cm TENG. FIG. 5B presents a brightfield image of the GDNF side of the 5 cm TENG. FIG. 5C presents an image of AAV2-GFP expression in the GFP side of the TENG. The GFP side of the TENG has robust expression of the GFP axonal signal. FIG. 5D presents an image of AAV2-GDNF expression on the GDNF side of the TENG. The GDNF side of the TENG expressed no GFP signal. FIG. 5E presents a graphical representation of the hGDNF concentrations of the AAV2-GFP group and the AAV2-GDNF group. Via ELISA, robust expression of GDNF from DRG transduced with AAV2-CMV-GDNF was detected (FIG. 5E). The results of FIG. 5A-FIG. 5E also demonstrated the successful transduction of two different AAV2 vectors on each end of the DRG, and the spatial distribution of transgene expression from the 5 cm TENG.

Conclusions: Successful spatial control was demonstrated over transgene expression in the TENG system by fabricating TENGs exhibiting preferential expression of GFP or mCherry at each end of the graft. These TENGs were grown 5 cm in length. GFP transgene expression was higher at one end, while mCherry transgene expression was higher at the other end. Robust expression of hGDNF from the transduced DRG transduced with AAV2-CMV-GDNF was also shown. Ultimately, the results indicated that TENGs could be produced that could provide an inducible GDNF gradient across a graft thereby allowing for accelerated recovery.

### Example 2: Doxycycline-Based Induction of Transgene Expression

The present example describes doxycycline-based induction of transgene expression. In particular, rat DRGs were transduced with AAV2-TRE-hGDNF. The DRGs were cultured with or without doxycycline for 3-days, the conditioned media was collected, and hGDNF concentration was measured using ELISA.

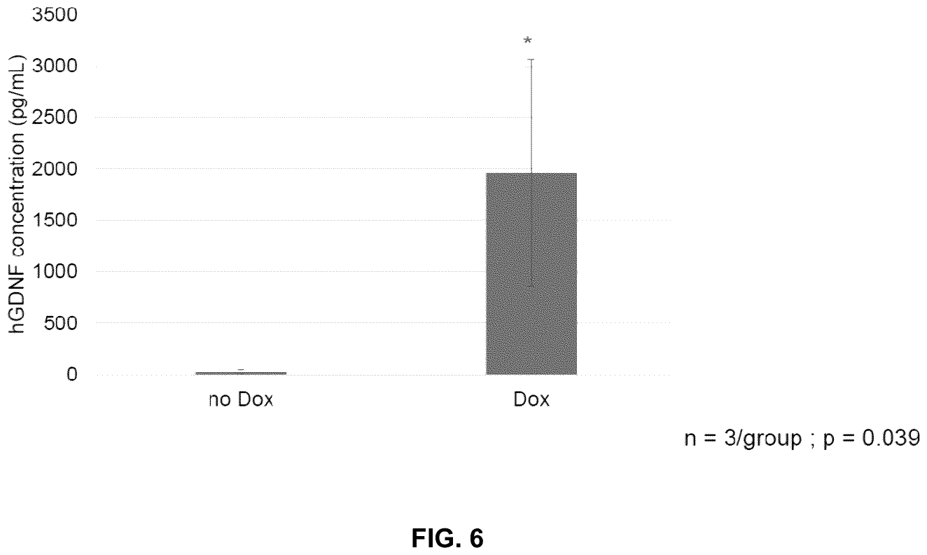

Referring now to FIG. 6, FIG. 6 presents a graphical representation of results related to doxycycline-based induction of transgene expression, where DRG explants were transduced with AAV2-TRE-hGDNF. A statistically significant increase in hGDNF production was detected in AAV2-TRE-hGDNF transduced rat DRGs with doxycycline added as compared to those cultured without doxycycline (p=0.039). The results demonstrated transgene expression from AAV-TRE-GDNF was controllable with doxycycline, and the results also demonstrated temporal control of growth factor secretion. For FIG. 6, n=3/group, and p=0.039.

### Example 3: Temporal Control of Transgene Expression of AAV-GDNF TENGs

The present example relates to the temporal control of transgene expression in AAV-GDNF TENGs. In particular, AAV-TENGs stretched to 1.5 cm length were cultured with doxycycline added on days 0-4. Doxycycline was then removed on days 4-20. GDNF levels in conditioned media were measured using ELISA.

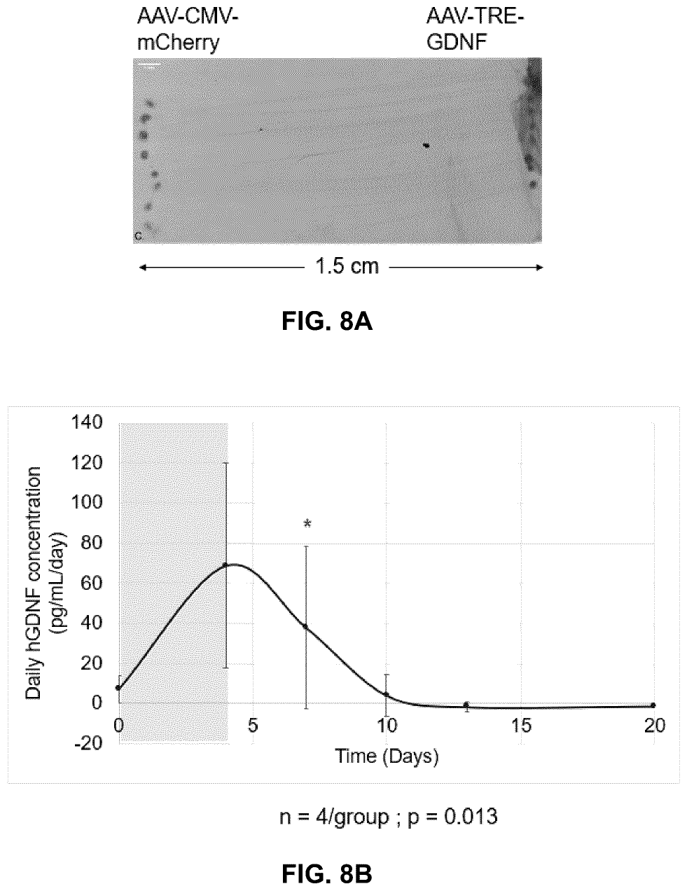

Referring now to FIG. 8A-FIG. 8B, FIG. 8A-FIG. 8B present images and graphical representations of results related to temporal control of AAV2-GDNF TENGs. FIG. 8A presents an image of an AAV-TENG stretch to 1.5 cm in length, with AAV2-CMV-mCherry on the left end, and AAV2-TRE-GDNF on the right end. FIG. 8B presents a graphical representation of the daily hGDNF concentration plotted against the time in days. Further referring to FIG. 8B, n=4/group, and p=0.013.

### Example 4: Spatial and Temporal Control of Transgene Expression of AAV-GDNF TENGs

The present example relates to the spatial and temporal control of transgene expression in AAV-GDNF TENGs. In particular, the AAV2-TRE-GDNF TENGs of Example 3 were taken at the end of day 20 and divided and cultured in media with doxycycline for 4 more days.

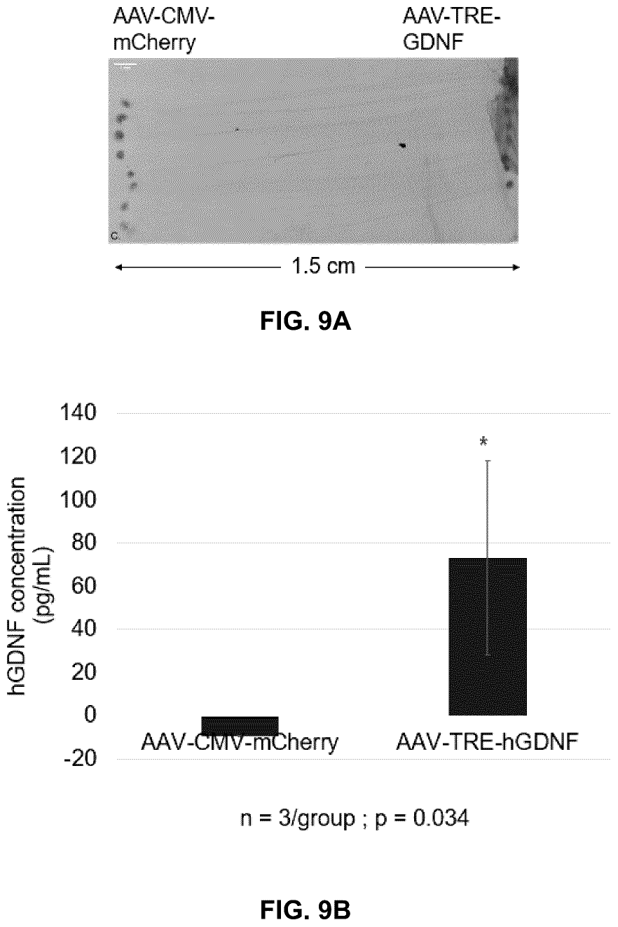

Referring now to FIG. 9A-FIG. 9B, FIG. 9A-FIG. 9B present images and graphical representations of results related to spatial and temporal control of AAV2-GDNF TENGs. FIG. 9A presents an image of an AAV-TENG stretched to 1.5 cm in length, with AAV2-CMV-mCherry on the left end, and AAV2-TRE-GDNF on the right end. FIG. 9B presents a graphical representation of the hGDNF concentration for each AAV2-CMV-mCherry and AAV2-TRE-hGDNF. N=3/group, and p=0.034. Further referring to FIG. 9B, there was statistically significant increase in GDNF level from the AAV2-TRE-GDNF ends versus the AAV2-CMV-mCherry end.

### Example 5: In Vivo Studies

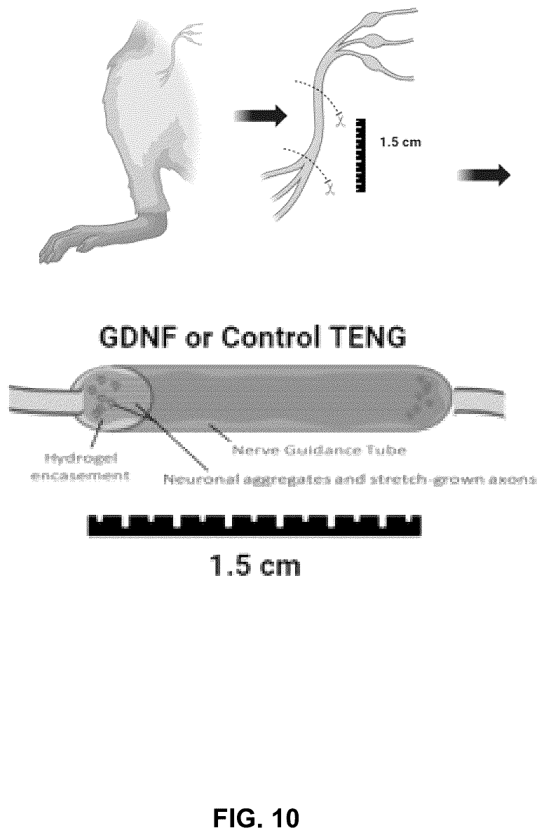

The instant example relates to in vivo studies using a rat sciatic nerve injury as a model system. For instance, FIG. 10 provides a schematic representation of an example of an in vivo study strategy using the rat sciatic nerve injury model.

For the studies in the present example, a 1.5 cm nerve defect was bridged with one of the following grafts: Group 1, AAV-TENG with constitutively active GDNF expression from the distal end of the graft; or Group 2, control TENG with no GDNF expression. Neuromuscular electrophysiology was assessed at 12- and 24-weeks post-repair, i.e. Compound Muscle Action Potential (“CMAP”). Nerve stimulation occurred proximal to the repair zone, and muscle response was recorded distal to the repair zone.

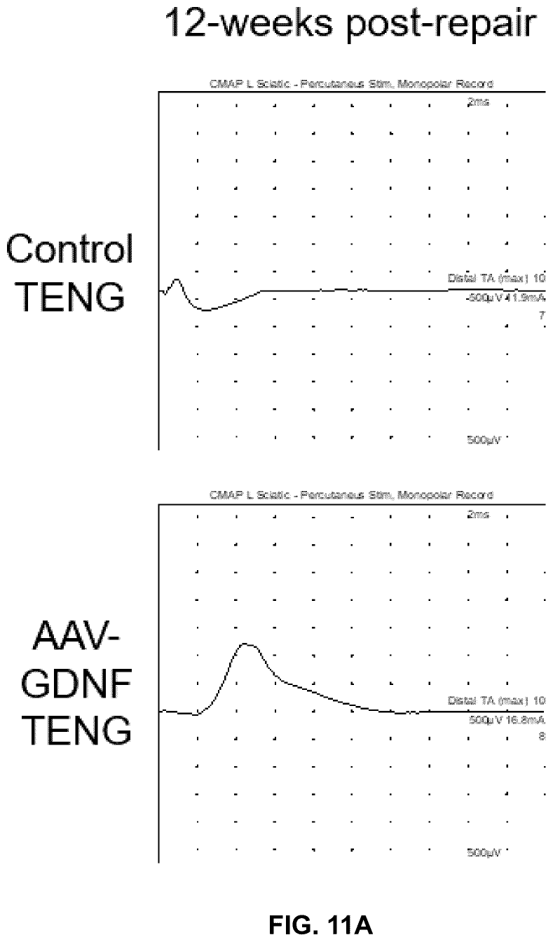

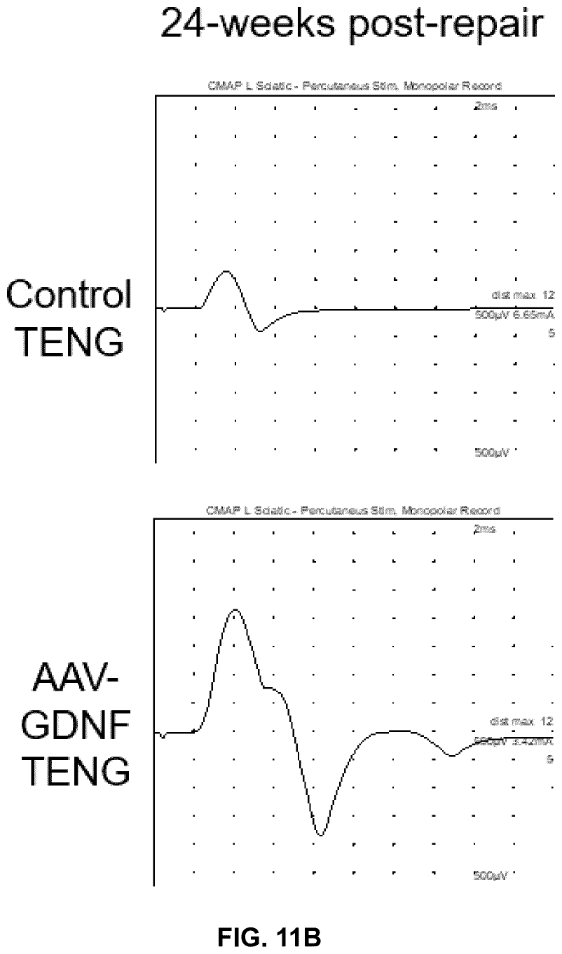

Referring now to FIG. 11A-FIG. 11B, FIG. 11A-FIG. 11B present graphical representations of results related to muscle response at 12 weeks (FIG. 11A) and 24 weeks (FIG. 11B) post repair for control TENG and AAV2-GDNF TENG groups. Measurable muscle response (see FIG. 11A-FIG. 11B) indicated axonal regeneration across the repair zone to reinnervate muscle. Furthermore, the results indicated a higher magnitude response following repair with AAV2-GDNF TENGs compared to standard control TENGs.

### Enumerated Embodiments

In some aspects, the present invention is directed to the following non-limiting embodiments:

Embodiment 1: A composition comprising tissue engineered nerve grafts (TENGs) modified to controllably express a neurotrophic growth factor.

Embodiment 2: The composition of embodiment 1, wherein the TENGs are modified to controllably express the neurotrophic growth factor under the control of an inducible promoter.

Embodiment 3: The composition of embodiment 2, wherein the inducible promoter is an inducible tetracycline response element (TRE) promoter.

Embodiment 4: The composition of embodiment 1, wherein the TENGs are modified to controllably express the neurotrophic growth factor at a first end of the TENG.

Embodiment 5: The composition of embodiment 1, wherein the TENGs are modified to controllably express the neurotrophic growth factor at a second end of the TENG.

Embodiment 6: The composition of embodiment 1, wherein the TENGs are modified to controllably express two different neurotrophic growth factors.

Embodiment 7: The composition of embodiment 1, wherein the TENGs are modified to controllably express a neurotrophic growth factor at a first end of the TENG, and to controllably express a neurotrophic growth factor at a second end of the TENG.

Embodiment 8: The composition of embodiment 7, wherein the neurotrophic growth factor at the first end of the TENG and the neurotrophic growth factor at the second end of the TENG are different neurotrophic growth factors.

Embodiment 9: The composition of embodiment 1, wherein the TENGs comprise an expression vector to controllably express the neurotrophic growth factor.

Embodiment 10: The composition of embodiment 9, wherein the expression vector is delivered by an adeno-associated viral (AAV) vector.

Embodiment 11: The composition of embodiment 10, wherein the AAV vector is an AAV2 vector.

Embodiment 12: The composition of embodiment 1, wherein the TENGs comprise a nucleic acid comprising the sequence of SEQ ID NO: 3 or SEQ ID NO: 4

Embodiment 13: The composition of embodiment 1, wherein the neurotrophic growth factor is selected from the group consisting of brain-derived neurotrophic factor (BDNF), nerve growth factor (NGF), and glial cell line-derived neurotrophic factor (GDNF).

Embodiment 14: The composition of embodiment 13, wherein the neurotrophic growth factor is GDNF.

Embodiment 15: The composition of embodiment 1, wherein the TENGs are stretch-grown TENGs.

Embodiment 16: A method of treating a nerve injury in a subject, wherein the method comprises contacting a site of nerve injury with a stretch-grown tissue engineered nerve graft (TENG) modified to controllably express a neurotrophic growth factor.

Embodiment 17: The method of embodiment 16, wherein the subject is a human.

Embodiment 18: The method of embodiment 16, wherein the nerve injury comprises an injury to a peripheral nerve of the subject.

Embodiment 19: The method of embodiment 16, wherein the nerve injury comprises the loss of a segment of nerve.

Embodiment 20: The method of embodiment 16, wherein the nerve injury comprises a nerve lesion of from about 1 cm to about 5 cm in length.

Embodiment 21: The method of embodiment 16, wherein the TENGs are modified to controllably express the neurotrophic growth factor under the control of an inducible promoter. Embodiment 22: The method of embodiment 16, wherein the TENGs are modified to controllably express the neurotrophic growth factor at a distal position of the nerve injury.

Embodiment 23: The method of embodiment 16, wherein the TENGs are modified to controllably express the neurotrophic growth factor at a proximal position of a nerve injury.

Embodiment 24: The method of embodiment 16, wherein the TENGs are modified to controllably express two different neurotrophic growth factors.

Embodiment 25: The method of embodiment 16, wherein the TENGs are modified to controllably express a neurotrophic growth factor at a distal position of a nerve injury, and to controllably express a neurotrophic growth factor at a proximal position of a nerve injury.

Embodiment 26: The method of embodiment 25, wherein the neurotrophic growth factor at the distal position of the nerve injury and the neurotrophic growth factor at the proximal position of the nerve injury are different neurotrophic growth factors.

Embodiment 27: The method of embodiment 16, wherein the TENGs comprise an expression vector to controllably express the neurotrophic growth factor.

Embodiment 28: The method of embodiment 27, wherein the expression vector is delivered by an adeno-associated viral (AAV) vector.

Embodiment 29: The method of embodiment 16, wherein the neurotrophic growth factor is selected from the group consisting of brain-derived neurotrophic factor (BDNF), nerve growth factor (NGF), and glial cell line-derived neurotrophic factor (GDNF).

Embodiment 30: The method of embodiment 29, wherein the neurotrophic growth factor is GDNF.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In sum, while this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising tissue engineered nerve grafts (TENGs) modified to controllably express a neurotrophic growth factor.

2. The composition of claim 1, wherein the TENGs are modified to controllably express the neurotrophic growth factor under the control of an inducible promoter.

3. The composition of claim 2, wherein the inducible promoter is an inducible tetracycline response element (TRE) promoter.

4. The composition of claim 1, wherein the TENGs are modified to controllably express the neurotrophic growth factor at a first end of the TENG.

5. The composition of claim 1, wherein the TENGs are modified to controllably express the neurotrophic growth factor at a second end of the TENG.

6. The composition of claim 1, wherein the TENGs are modified to controllably express two different neurotrophic growth factors.

7. The composition of claim 1, wherein the TENGs are modified to controllably express a neurotrophic growth factor at a first end of the TENG, and to controllably express a neurotrophic growth factor at a second end of the TENG.

8. The composition of claim 7, wherein the neurotrophic growth factor at the first end of the TENG and the neurotrophic growth factor at the second end of the TENG are different neurotrophic growth factors.

9. The composition of claim 1, wherein the TENGs comprise an expression vector to controllably express the neurotrophic growth factor.

10. The composition of claim 9, wherein the expression vector is delivered by an adeno-associated viral (AAV) vector.

11. The composition of claim 10, wherein the AAV vector is an AAV2 vector.

12. The composition of claim 1, wherein the TENGs comprise a nucleic acid comprising the sequence of SEQ ID NO: 3 or SEQ ID NO: 4.

13. The composition of claim 1, wherein the neurotrophic growth factor is selected from the group consisting of brain-derived neurotrophic factor (BDNF), nerve growth factor (NGF), and glial cell line-derived neurotrophic factor (GDNF).

14. The composition of claim 13, wherein the neurotrophic growth factor is GDNF.

15. The composition of claim 1, wherein the TENGs are stretch-grown TENGs.

16. A method of treating a nerve injury in a subject, wherein the method comprises contacting a site of nerve injury with a stretch-grown tissue engineered nerve graft (TENG) modified to controllably express a neurotrophic growth factor.

17. The method of claim 16, wherein the subject is a human.

18. The method of claim 16, wherein the nerve injury comprises an injury to a peripheral nerve of the subject.

19. The method of claim 16, wherein the nerve injury comprises the loss of a segment of nerve.

20. The method of claim 16, wherein the nerve injury comprises a nerve lesion of from about 1 cm to about 5 cm in length.

21. The method of claim 16, wherein the TENGs are modified to controllably express the neurotrophic growth factor under the control of an inducible promoter.

22. The method of claim 16, wherein the TENGs are modified to controllably express the neurotrophic growth factor at a distal position of the nerve injury.

23. The method of claim 16, wherein the TENGs are modified to controllably express the neurotrophic growth factor at a proximal position of a nerve injury.

24. The method of claim 16, wherein the TENGs are modified to controllably express two different neurotrophic growth factors.

25. The method of claim 16, wherein the TENGs are modified to controllably express a neurotrophic growth factor at a distal position of a nerve injury, and to controllably express a neurotrophic growth factor at a proximal position of a nerve injury.

26. The method of claim 25, wherein the neurotrophic growth factor at the distal position of the nerve injury and the neurotrophic growth factor at the proximal position of the nerve injury are different neurotrophic growth factors.

27. The method of claim 16, wherein TENGs comprise an expression vector to controllably express the neurotrophic growth factor.

28. The method of claim 27, wherein the expression vector is delivered by an adeno-associated viral (AAV) vector.

29. The method of claim 16, wherein the neurotrophic growth factor is selected from the group consisting of brain-derived neurotrophic factor (BDNF), nerve growth factor (NGF), and glial cell line-derived neurotrophic factor (GDNF).

30. The method of claim 29, wherein the neurotrophic growth factor is GDNF.


SEQUENCE LISTING (6 sequences)

SEQ ID NO: 1 - DNA, 5118 bp, synthetic construct
  source 1..5118: mol_type = other DNA; note = AAV2-CMV-GFP
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 2 - DNA, 5109 bp, synthetic construct
  source 1..5109: mol_type = other DNA; note = AAV2-CMV-mCherry
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 3 - DNA, 4481 bp, synthetic construct
  source 1..4481: mol_type = other DNA; note = AAV2-CMV-GDNF
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 7530 bp, synthetic construct
  source 1..7530: mol_type = other DNA; note = AAV2-TRE-GDNF
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacatggggccgggattttcctccacgtccccgcatgttagaagacttcccctgccctctccggagccccagggatcctctccttgctgcaacagggagatcacctttggtttggtaaacaggaatcctgccatggaggccaggttgctgtaattctccagcatcacctcacggtacaggcttctctgagacagatccagcttcttccactcgtcccgagtaaagagcacagccacatcttcaaatgtcactgacactgctagctttctcttcttttttggtttattaccatcctcaatgggtgtatgctttttttcatctaactgtgcagaaaatccaaatattaatgatttcagcccgaaatggaaggccatttcagcagaggttctcgcctgaatattaaatgcctcttgtaataatggtggtgcagcgtctatcacatgattattttctatctggtttgttgcttgctcctctaatactgcacccaacgtaaaatggctgatagattgcagaatgaaaagagcctcctcgaccgaaaaccctgcatcgcatagacagcgtagttgcgcctctgcttgttcaaactgggggggcgtaggagaggtccctatatgcaatcgggctccatcacgatggaccagtaatgctttacggaaactcagagcattttcctggagaaactgctgccaactctcagtcggtaacggtgctgaacgggtgtgatgcttcgccagtattgcctctgaaagcatgttcataagagtctgcttgttgcgcacgtgccaatacaatgtaggctgctctacacctagcttctgggcgagtttacgggttgttaaaccttcgattccgacctcattaagcagctctaatgcgctgttaatcactttacttttatctaatctagacatgaattcccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacgagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattg

SEQ ID NO: 5 - DNA, 687 bp, synthetic construct
  source 1..687: mol_type = other DNA; note = Synthetic Human GDNF
atgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 6 - protein, 228 aa, synthetic construct
  source 1..228: note = Synthetic Human GDNF amino acid sequence
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI